(19) United States
(12) Patent Application Publication
(10) Pub. No.: US 2010/0160717 A1
(43) Pub. Date: Jun. 24, 2010

(54) In vitro fertilization

(76) Inventors: Richard T. Scott, JR., Far Hills, NJ (US); Nathan R. Treff, Chester, NJ (US)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 12/587,406

(22) Filed: Oct. 5, 2009

Related U.S. Application Data
(60) Provisional application No. 61/195,223, filed on Oct. 3, 2008.

(51) Int. Cl. A61B 17/435; C12Q 1/68

(52) U.S. Cl. 600/34; 435/6

(57) ABSTRACT

Methods of in vitro fertilization wherein said method includes preimplantation genetic diagnosis of all 24 chromosomes of an IVF embryo comprising whole genome amplification and SNP-based microarray analyses are disclosed.

This application claims the benefit of U.S. Provisional Application No. 61/195,223, filed Oct. 3, 2008, which is incorporated by reference in its entirety herein.

### BACKGROUND OF THE INVENTION

The high rate of multiple births resulting from in vitro fertilization or IVF is a significant problem in the industry. Over 100,000 in vitro fertilization procedures are performed in the U.S. each year, and while multiple fertilized IVF embryos are reintroduced, only about ⅓ of them result in successful pregnancies. And a high rate of those successful pregnancies resulted in multiple births. The main reason for multiple gestations following in vitro fertilization is an inability to precisely estimate the reproductive potential of individual embryos; thus the typical in vitro fertilization procedure typically involves the transfer of multiple embryos in the hopes that at least one of them will lead to pregnancy. Accordingly, techniques for prequalifying embryos for implantation are highly desirable. Indeed, ensuring that all of the embryos transferred have a maximum chance of resulting in normal, healthy children would reduce the number of IVF embryos necessary for transfer, increase the percentage of births, decrease the chance of genetic birth defects such as Down's Syndrome, and reduce miscarriage, reduce multiple gestations and, overall, reduce the need to transfer multiple, fertilized embryos. IVF embryo prescreening is typically performed in a procedure referred to as “preimplantation genetic diagnosis” (PGD). Methods for PGD include, inter alia, the use of proton NMR to determine the metabolic profile of an embryo, as well as genetic testing of embryos for chromosomal abnormalities using SNPs and microarrays. See WO2007/070482 and US2008/0085836.

The genomes of all organisms undergo spontaneous mutation in the course of their continuing evolution, generating variant forms of progenitor sequences (Gusella, Ann. Rev. Biochem. 55, 831-854 (1986)). The variant form may confer an evolutionary advantage or disadvantage relative to a progenitor form or may be neutral. In some instances, a variant form confers a lethal disadvantage and is not transmitted to subsequent generations of the organism. In other instances, a variant form confers an evolutionary advantage to the species and is eventually incorporated into the DNA of many or most members of the species and effectively becomes the progenitor form. Additionally, the effect of a variant form may be both beneficial and detrimental, depending on the circumstances. For example, a heterozygous sickle cell mutation confers resistance to malaria, but a homozygous sickle cell mutation is usually lethal. In many instances, both progenitor and variant form(s) survive and co-exist in a species population. The coexistence of multiple forms of a sequence gives rise to polymorphisms.

Approximately 90% of all polymorphisms in the human genome are single nucleotide polymorphisms (SNPs). SNPs are single base pair positions in DNA at which different alleles, or alternative nucleotides, exist in some population. The SNP site is often preceded by and followed by highly conserved sequences of the allele (e.g., sequences that vary in less than 1/100 or 1/1000 members of the populations). An individual may be homozygous or heterozygous for an allele at each SNP position.

A SNP may arise due to a substitution of one nucleotide for another at the polymorphic site. Substitutions can be transitions or transversions. A transition is the replacement of one purine nucleotide by another purine nucleotide, or one pyrimidine by another pyrimidine. A transversion is the replacement of a purine by a pyrimidine, or vice versa. A SNP may also be a single base insertion/deletion variant (referred to as “indels”). A substitution that changes a codon coding for one amino acid to a codon coding for a different amino acid is referred to as a non-synonymous codon change, or missense mutation. A synonymous codon change, or silent mutation, is one that does not result in a change of amino acid due to the degeneracy of the genetic code. A nonsense mutation is a type of non synonymous codon change that results in the formation of a stop codon, thereby leading to premature termination of a polypeptide chain and a defective protein.

SNPs, in principle, can be bi-, tri-, or tetra-allelic. However, tri- and tetra-allelic polymorphisms are extremely rare, almost to the point of non-existence (Brookes, Gene 234 (1999) 177-186). For this reason, SNPs are often referred to as “bi-allelic markers” or “di-allelic markers”.

SNPs are useful in association studies for identifying particular SNPs, or other polymorphisms, associated with pathological conditions, such as human disease. Association studies may be conducted within the general population and are not limited to studies performed on related individuals in affected families (linkage studies). An association study using SNPs involves determining the frequency of the SNP allele in many patients with the disorder of interest, such as human disease, as well as controls of similar age and race. The appropriate selection of patients and controls is critical to the success of SNP association studies. Therefore, a pool of individuals with well-characterized phenotypes is extremely desirable. For example, blood pressure and heart rate can be correlated with SNP patterns in hypertensive individuals in whom these physiological parameters are known in order to find associations between particular SNP genotypes and known phenotypes.

Significant associations between particular SNPs or SNP haplotypes and phenotypic characteristics can be determined by standard statistical methods. Association analysis can either be direct or “linkage disequilibrium” or “LD” based. In direct association analysis, causative SNPs are tested that are candidates for the pathogenic sequence itself.

In LD based SNP association analysis, random SNPs are tested over a large genomic region, possibly the entire genome, in order to find a SNP in LD with the true pathogenic sequence or pathogenic SNP. For this approach, high density SNP maps or arrays are required in order for random SNPs to be located close enough to an unknown pathogenic locus to be in linkage disequilibrium with that locus in order to detect an association. SNPs tend to occur with great frequency and are spaced uniformly throughout the genome. The frequency and uniformity of SNPs means that there is a greater probability, compared with other types of polymorphisms, such as tandem repeat polymorphisms, that a SNP will be found in close proximity to a genetic locus of interest. SNPs are also mutationally more stable than tandem repeat polymorphisms, such as variable number tandem repeat polymorphisms (VNTRs). LD-based association studies are capable of finding a disease susceptibility gene without any a priori assumptions about what or where the gene is. See U.S. Pat. No. 6,812,339.

### SUMMARY OF THE INVENTION

In one aspect, the present invention relates to a method of in vitro fertilization wherein said method includes preimplantation genetic diagnosis and selection of an IVF embryo comprising the steps of:
(a) biopsying said IVF embryo to remove one or more cells from said embryo;
(b) extracting nucleic acid from said one or more cells;
(c) performing whole-genome amplification of said nucleic acid;
(d) gathering genetic information for said IVF embryo comprising performing the following substeps (i) and (ii):
   i) determining the copy number of one or more SNPs in said IVF embryo; and
   ii) determining the copy number of one or more chromosomes in said IVF embryo; and optionally
   iii) determining the presence of a loss or gain in chromosomal heterozygosity in said IVF embryo;
(e) predicting the genetic normalcy of the IVF embryo based on the genetic information obtained in (d) if the genetic information collected in (d)(i) indicates at least 90% SNP copy number concurrence, and the genetic information collected in (d)(ii) indicates at least a 50% chromosomal copy number concordance and where applicable the genetic information collected in (d)(iii) indicates neither a gain nor loss of chromosomal heterozygosity; and
(f) selecting one or more IVF embryos for transfer based on the prediction made in step (e).

In particular embodiments, it is contemplated herein that polar bodies, blastomeres, or trophectoderm may be biopsied. It is also contemplated that in particular embodiments, the biopsy is taken from an embryo at about day 0 to about day 6 of development. In another embodiment, predicting the genetic normalcy of an IVF embryo comprises determination of the karyotype of said embryo. In a further embodiment, it is contemplated herein that the copy number of one or more SNPs for all 24 chromosomes is determined and/or the copy number of all 24 chromosomes in the embryo is determined. In an additional embodiment, the method of the present invention includes determination of the copy number of about 250,000 or more SNPs. It yet another embodiment, the nucleic acid is DNA.

In another embodiment, the method of the invention further comprises transferring one or more IVF embryos selected according to step (f). In a particular embodiment, one or more IVF embryos are transferred within about 48 hours to about 72 hours of embryo biopsy. In another embodiment, said one or more IVF embryos are frozen prior to transfer. In an additional embodiment, the transfer is a “fresh transfer”, i.e., the embryo is not frozen prior to transfer.

In yet another embodiment, it is contemplated herein that the genetic information determined for each individual IVF embryo is recorded prior to transfer.

A further embodiment of the method contemplates that all substeps (d)(i), (d)(ii) and (d)(iii) are performed.

In a second aspect, the invention relates to a method of identifying the genetic characteristics of an IVF embryo likely to result in successful embryo transfer and live birth comprising
(a) gathering genetic information from a child resulting from a successful pregnancy achieved according to the methods of the present invention;
(b) comparing said genetic information to the recorded data for each IVF embryo transferred, in order to identify the IVF embryo that implanted and resulted in the live birth; and
(c) determining whether assumptions and observations made which led to the selection of said embryo for transfer were correct.

In another aspect, the invention relates to a method for identifying a specific IVF embryo which implanted and resulted in a successful pregnancy and live birth of a child comprising
(a) gathering genetic information from the child;
(b) comparing said genetic information to the genetic information recorded for each IVF embryo transferred, and
(c) using the recorded genetic information for each transferred embryo as a “genetic fingerprint” by which the child may be linked to the correct transferred embryo.

In yet another aspect, the present invention relates to a method of in vitro fertilization which permits the identification of transferred and implanted IVF embryo(s) comprising performing in vitro fertilization according to the methods of the present invention and further comprising gathering genetic information from a resulting child or children and comparing with genetic information previously recorded for each transferred embryo.

### BRIEF DESCRIPTION OF THE DRAWINGS

FIG. 1 illustrates single nucleotide polymorphism in DNA.

FIG. 2 illustrates lysis of a blastomere to obtain DNA for WGA analysis.

FIG. 3 illustrates an overview of the WGA amplification process.

FIG. 4 is an illustration, adapted from the illustrations from Affymetrix showing the overall process up to analysis including digestion, amplification, and hybridization of DNA including the attachment of the amplified DNA fragments to a microarray chip.

FIG. 5 illustrates the raw signal data and copy number analysis resulting from WGA analysis.

FIG. 6 illustrates the copy number analysis done on cells from a normal female.

FIG. 7 illustrates data generated in a heterozygosity analysis.

FIG. 8 illustrates the predictive value of microarray-based 24 chromosome aneuploidy screening. Data indicate that euploid embryos implant nearly twice as often as mixed ploidy embryos indicating excellent positive predictive value. In addition, aneuploidy embryos have 0% sustained implantation indicating excellent negative predictive value. (See Example 3).

FIG. 9 illustrates data from embryos leading to the first euploid embryo selection in a fresh embryo transfer and delivery of healthy twin boys. Ten embryos (E1-10) were biopsied for microarray analysis of a single blastomere on day 3. Results were obtained on day 5 of embryo development and embryos 9 and 10 were selected for fresh transfer. Newborn buccal DNA was profiled and compared with the original blastomere biopsy for confirmation through DNA fingerprinting. (See Example 4).

FIG. 10 illustrates whole genome amplification (WGA) DNA behaves differently from unamplified gDNA. Polymerase chain reaction (PCR)-based microarray target preparation results in less (A) and smaller-sized (B) DNA (a representative agarose gel image) from pooled human lymphocyte-derived genomic DNA (gDNA) compared with single human lymphocyte WGA-derived DNA. In addition, heterozygous call rates are significantly reduced in single lymphocyte WGA-derived DNA compared with gDNA (C). StyI and NspI represent both the restriction endonuclease used to prepare target DNA and the microarray used to characterize 238,304 and 262,264 SNPs, respectively. Error bars ±1 standard deviation. (See Example 6).

FIG. 11 illustrates that single-lymphocyte and unamplified genomic DNA (gDNA) relationships can be determined with 100% sensitivity and specificity. (A) Using the default settings, genotype similarities are lower for sibling comparisons than for match comparisons of single lymphocytes and unamplified gDNA (default 250K analysis); the separation of match and sibling similarities improves with increased Gtype stringency from 0.33 to 0.01 (increased 250K call stringency), and dramatically improves with the use of parental genomic DNA to identify informative single nucleotide polymorphisms (SNPs) (stringent and informative 250K); no improvement was observed by increasing the number of SNPs analyzed (stringent and informative 500K). (B) The number of similar SNPs (of the total possible comparisons) decreased with additional stringency, informativeness, and number of SNPs analyzed. (C) Finally, the diagnostic value of single-lymphocyte DNA fingerprinting was evaluated by receiver operating characteristic curve analysis and demonstrated 100% sensitivity and specificity for a range of similarity thresholds described in the text. The black bar in panel B, under stringent and informative 250K, reflects a threshold that could be applied to predict the relationship of single blastomeres to unamplified fetal gDNA for clinical research (embryo tracking). (See Example 6).

FIG. 12 illustrates the relationship of single human research blastomeres with other single blastomeres can be determined with 100% sensitivity and specificity. (A) Whole genome amplification (WGA) yield from three cohorts of single blastomeres was sufficient from 68 out of 72 attempts. (B) The total single-nucleotide polymorphism (SNP) calls and heterozygous SNP calls, from the NspI 250K microarray, were significantly reduced (P<0.05) in single blastomeres (n=47) compared with single lymphocytes (n=20). (C) Distinct separation of matched and sibling single blastomere relationships is achieved using WGA and stringent and informative SNPs from the NspI 250K SNP microarray. A range of thresholds, similar to lymphocytes and described in the text, perform with 100% sensitivity and specificity in determining the relationships of single blastomeres with other single blastomeres. (See Example 6).

FIG. 13 illustrates an example of molecular karyotyping comparison results of villi from products of conception (POC) and blastomeres from embryos transferred in an IVF cycle. One blastomere from each of five embryos (four that were transferred and one that was cryopreserved) was karyotyped by copy number analysis of 24 chromosomes and compared with the karyotype obtained from the POC villi tissue after clinical pregnancy loss. The karyotype is indicated within each copy number graph. The percentage genotype similarity of each blastomere to the POC villi is shown in parentheses. The y-axis is copy number state (labeled as 1 through 4 copies), and the x-axis is chromosome number. Percent similarities of transferred embryos assigned as siblings by fingerprinting were similar to percentages obtained for cryopreserved embryos (known siblings). (See Example 6).

FIG. 14 illustrates a summary of clinical case DNA fingerprinting and karyotype-based match with products of conception (POC) tissues. (See Example 6).

### DETAILED DESCRIPTION

By concurrence or concordance, used interchangeably herein, it is meant the rate at which SNPs on each chromosome are assigned the same copy number state.

As used herein, “embryo biopsy” is meant to include removal of cells (blastomeres) from the embryo, or polar bodies or trophectoderm associated with the embryo. It is contemplated herein that biological material may be similarly biopsied from an oocyte and analyzed according to the methods of the present invention.

As referred to herein, gathering genetic information from a child includes sampling biologic material from a child prenatally or postnatally.

As used herein, “nucleic acid” includes, but is not limited to, DNA.

As referred to herein, genetic information may be “recorded”, e.g., noted and memorialized for future reference, according to conventional scientific methods and practices familiar to one of skill in the art.

Women undergoing IVF require hormone injections to stimulate follicular development and multiple egg production. This stimulation process usually requires the initial use of a gonadotropin releasing hormone (GnRH) agonist to suppress ovarian function, preventing ovulation until the desired time. Protocols for these injections are well known.

At the appropriate time, unfertilized eggs are harvested. Egg retrieval involves placing a special needle into the ovarian follicle and removing the fluid that contains the egg, again using known techniques. Once the follicular fluid is removed from the follicle, the eggs are inspected microscopically and placed into an incubator. Conventional insemination or intracytoplasmic sperm injection (ICSI) is used to fertilize the eggs. The type of fertilization employed is based on the male's semen parameters and/or the type of analysis required. ICSI is preferred for all testing employing microarray analysis or DNA sequencing.

During conventional insemination, sperm are mixed with each egg in a culture dish and incubated overnight to undergo the fertilization process. Intracytoplasmic sperm injection is a technique whereby one sperm is directly injected into one egg. With either technique, the eggs are checked the day after to evaluate for early cell division. The fertilized eggs are now called embryos and are placed in a special culture media to promote growth and development. On day-3 of development (three days after retrieval) one or two blastomeres are removed from each cleaving embryo by a procedure called Embryo Biopsy for genetic testing. Three basic biopsy techniques are employed by those of skill in the art. These techniques include mechanically creating an incision in the zona pellucida using a specialized microsurgical knife or glass needle; chemically digesting a portion of the zona pellucida with acid Tyrode's solution; or removing the zona pellucida using a laser beam. All three pose significant risks of damaging a day-3 embryo during biopsy with a subsequent reduction in implantation rates, a possible increase in biochemical pregnancies and a reduction in the birth of healthy, normal babies. One recent scientific publication suggested that day-3 embryo biopsies induce significant damage to the in vitro developing embryo and reduces implantation by approximately 30% (Mastenbroek et al. N Engl J Med. 2007 Jul. 5; 357(1):9-17). While others disagree with this risk, nevertheless, steps should be taken to reduce the risk of significantly damaging the embryo during the biopsy procedure.

Ideally, IVF embryos are analyzed, graded and ranked for potential transfer. Typically, embryos to be transferred are placed through the cervix into the uterine cavity using a small, soft catheter. This procedure usually requires no anesthesia.

As expected, embryo grading and ranking have played significant roles in the IVF process, as clinicians have strived to identify “the best” embryo(s) for transfer, i.e., those which are most likely to achieve a viable pregnancy. To this end, grading and ranking of IVF embryos typically includes not only microscopic physical analysis of the embryo to identify those embryos which appear to be developing normally, but also genetic analysis of the embryo (preimplantation genetic diagnosis, or PGD) to identify whether the embryo contains chromosomal abnormalities. With regard to the latter, as reported herein, grading and ranking of IVF embryos for transfer may now include genomic analysis of the embryo using a whole genome amplification and single nucleotide polymorphism (SNP)-based microarray paradigm as a means to provide an accurate and reliable single cell 24 chromosome aneuploidy screening technology.

Cells contain chromosomes, which are string-like structures where genetic material resides. The genetic material is called a gene. Genes are made up of DNA sequences. Each cell has approximately 25,000 genes. Cells also contain mitochondrial organelles that contain a different type of DNA.

Genetic disease is caused by abnormalities of gene function. This can occur by having too many or too few chromosomes (aneuploidy), when chromosome pieces are attached to the wrong chromosome (translocation), when one is missing or containing an extra piece of a chromosome (deletion or duplication), when part of a chromosome is upside down (inversion), or when the genomic (nuclear) or mitochondrial DNA sequence is changed. In order to perform PGD for the analysis and grading of an IVF embryo, nucleic acid, e.g., DNA, must be collected from the embryo by biopsy, replicated in vitro many times and analyzed.

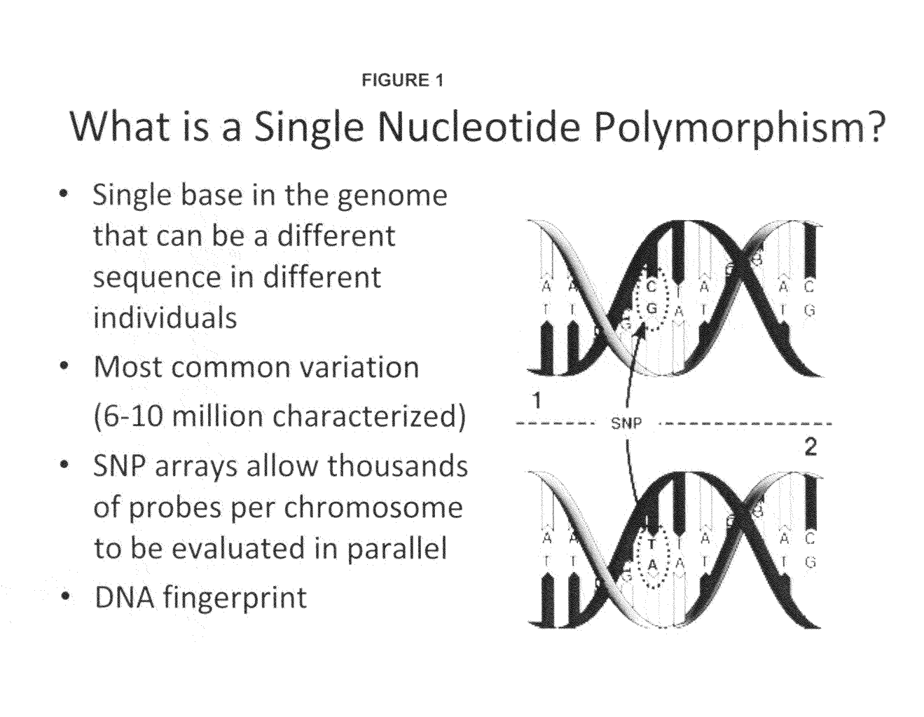

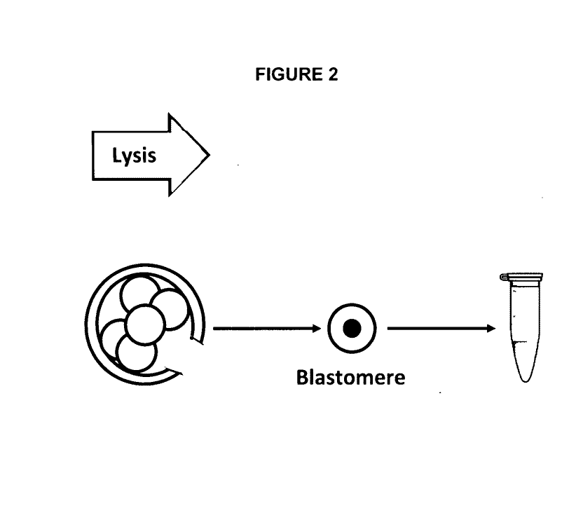

As contemplated herein, PGD involves using SNPs in a microarray analysis. SNPs were discussed previously and are illustrated in FIG. 1. Simply put, a microarray provides a platform upon which millions of individual assays may be performed simultaneously. Indeed, the massive data set that is generated from a single microarray experiment has changed the focus of scientific experimental design and medical diagnoses. Because millions of genetic variations may be tested at one time by one microarray, the rational approach of generating “if-then” hypotheses derived from prior system knowledge have been obviated in favor of a data driven, hypothesis-free approach. Hence, unknown genetic samples may be analyzed for a host of human diseases, syndromes, and phenotypic states.

Microarrays, also referred to as microchip arrays, arrays or biochips, have been widely used for gene expression and other genomic research. The features of high density, flexible design, uniform hybridization efficiency, and massively parallel detection are but a few of their superior characteristics. From published genomic information, probes for microarray analysis can, be flexibly designed at any position along chromosomes for specific SNPs. Oligonucleotide DNA or RNA probes are readily manufactured at high density. Carefully selected and designed probes printed or synthesized on microarray chips can detect chromosome copy number, chromosome arrangement, and other abnormalities. These features provide technical advantages over the traditional bacterial artificial chromosome (BAC) array CGH and others like it.

As contemplated herein, microarrays disclosed herein may employ oligonucleotides designed to assess not only the whole chromosomal structure but also the finer chromosomal changes including aneuploidies, translocations, insertion, deletion, reversion, local amplification, and even single nucleotide polymorphisms.

The basics of a microarray analysis are well known. A microarray is generally made by printing or synthesizing nucleic acid that is complementary to known sequences in a genome onto a surface. The entire genome of an organism (e.g., humans), can be evaluated by hybridizing amplified and fluorescently labeled DNA to the array. To do this one must first determine where the genes are in the sequence, identify primer pairs that can be used in polymerase chain reaction or PCR to make copies of every gene, and replicate the genes and therefore their DNA, many, many times to increase the amount of genetic material. This is known as amplification. An important aspect of amplification is fidelity, that is, making sure that all of the copies of DNA made during amplification faithfully maintain the sequence, stoichiometry, and content of base pairs found in the DNA from the original DNA.

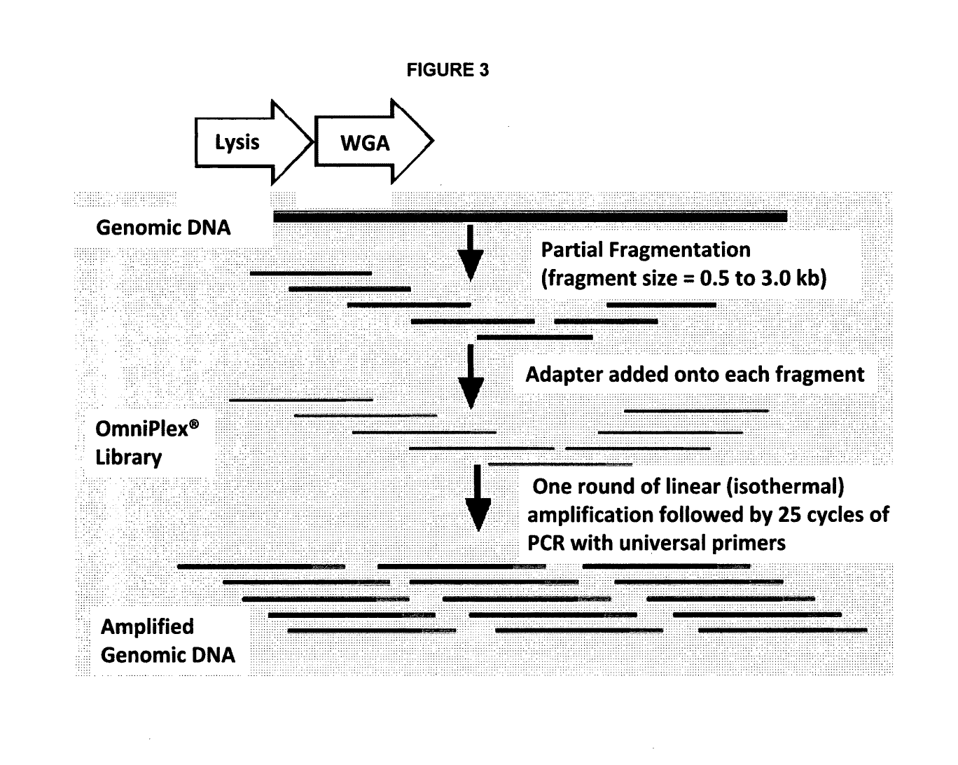

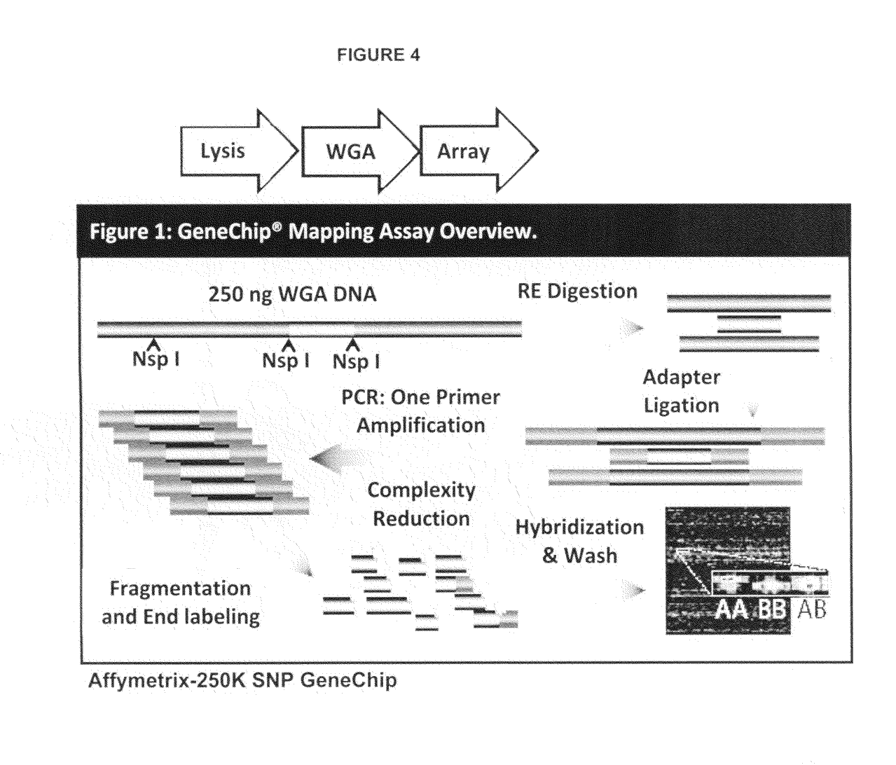

Using restriction enzymes, the DNA obtained from cells is chopped into smaller pieces at known cleavage sites, an adapter sequence is ligated to the restriction site “sticky ends,” PCR is performed using primers complementary to the adapter sequence, DNA is purified, fragmented, labeled, and then applied to the microchip array or microarray. See, FIGS. 3 and 4. See also GeneChip® Mapping 500K Assay Manual, Rev. 3 from Affymetrix, ©2005-06, the contents of which are incorporated by reference. The DNA fragments on the array are hybridized, annealed or reacted to those fragments from the DNA of the embryo such that complimentary base pairs of the DNA from the embryo will bind to or pair with the DNA immobilized on the microarray chip. Ideally some of the DNA, either that used to generate the microarray or, more typically, that from the embryo, is labeled with a material which can be detected and counted. See FIG. 4.

The more there is of a particular genetic sequence, a SNP in this case, the more will bind to an individual site on the microarray and that will translate into a particular color intensity, density of color, or some other property which is detectable and measurable and indicative of the presence, absence, and quantity of a particular fragment of DNA and/or particular SNP of the embryo. Terminal deoxynucleotidyl transferase may be used in an “end labeling” procedure where biotinylated nucleotides are added to the ends of the DNA fragments. (See GeneChip® Mapping Assay Manual previously incorporated by reference.)

A computer database lists the SNPs which are contained in each spot of the microarray. The labeled DNA fragments are added to the array where they hybridize to the complementary DNA on the microarray. The microarray is then washed to remove material that does not hybridize, stained, and scanned. Multiple scans can be run for each gene or gene section on the microarray and for each SNP determining the number of labeled complementary strands from the embryo that have attached at that point. Often this is judged through the intensity.

According to published application US2008/0085836, the largest hurdle in performing WGA on a single cell is getting enough DNA without introducing experimental artifact. Only approximately 6 picograms of genomic (nuclear) DNA exist within a single human blastomere or trophectoderm cell. In order to run a microarray analysis, one requires approximately 250 nanograms to successfully complete the assay. Therefore, one must incorporate additional DNA amplifications to attain the required amount of genomic DNA. US2008/0085836 suggests that PCR can be employed using universal primers to attain the required amount of DNA. However, according to those applicants, this PCR methodology induces experimental artifacts that result in preferential regions of amplification and/or deletion and/or other sequence specific issues.

PCR exploits the physical properties of the naturally occurring DNA polymerase from the thermophilic bacteria Thermophylis aquaticus (Taq) to remain functional at high temperatures. This Taq polymerase is used in an iterative process of DNA replication in vitro. PCR is typically considered to have three steps to the process: 1) DNA denaturation, 2) primer annealing, and 3) chain elongation. For DNA denaturation, the assay temperature is brought to about 95° C. to disrupt the hydrogen bonds between the nitrogenous bases of the nucleic acid secondary structure. Once denatured, the assay reaction temperature is reduced to a temperature that is sufficiently low enough for short, sequence-specific oligonucleotides to hybridize to the denatured genomic DNA recreating a local secondary structure, usually about 62° C. During chain elongation, the reaction temperature is raised to about 72° C., the optimal temperature for Taq polymerase, and the hybridized primer is extended as a function of polymerase fidelity. These three steps are repeated in an iterative, programmatic assay controlled by a thermocycler. While some suggest that PCR can preferentially amplify or fail to amplify genomic DNA, and therefore should not be used for DNA amplification from single cells (see US2008/008536), as now reported herein, Applicants have found that PCR may be considered a reliable and preferred method of amplification for PGD if done in accordance with the criteria set forth hereinbelow.

It has now been found that PCR can be used successfully in PGD as a way of generating sufficient DNA from a single cell for WGA and PGD using SNPs and microarray technology when performed according to the methods of the present invention. Specifically, as disclosed herein in detail, a highly validated WGA and SNP-based microarray paradigm can be used to provide accurate and reliable PGD by evaluating specific concordance strategies and Gaussian smoothing parameters as discussed in detail herein. For example, as contemplated herein, SNP copy number determination may be deemed reliable and useful for PGD, when the degree of concordance is at least 90% or better, and in another embodiment, at least 94% or better, and when the call rate (the number of times that a particular genotype is assigned a call as being homozygous AA, homozygous BB, heterozygous AB) is greater than 10% using a stringency of 0.01. At these parameters, it has been discovered that the fidelity of the PCR amplification is excellent and the microarray data is capable of being reliably used for PGD. In addition, SNP microarray data may be analyzed for copy number determination employing a Gaussian smoothing distance of 5 MB to accurately evaluate karyotype. (See Example 1).

In addition to PCR, other means of nucleic acid amplification for microarray analyses exists, and these are familiar to one of skill in the art, e.g., multiple displacement amplification (MDA), (Dean et al., Proc Natl Acad Sci USA. 2002 Apr. 16; 99(8):5261-6) ligase chain reaction (LCR) (Landegren Science 1988; 241:1077;), transcription amplification (Kwoh Proc. Natl. Acad. Sci. USA 1989; 86:1173); self-sustained sequence replication (Guatelli Proc. Natl. Acad. Sci. USA 1990; 87: 1874); Q Beta replicase amplification (Smith J. Clin. Microbial. 1997; 35:1477-1491), and other RNA polymerase mediated techniques such as nucleic acid sequence based amplification, or NASBA (Sambrook; Ausubel; U.S. Pat. Nos. 4,683,195 and 4,683,202). It is contemplated herein that by applying the concordance and additional parameter strategies as described herein to these additional amplification methods, they may also be suitable for use in PGD. Nevertheless, the ability to be able to rely on the use of PCR in PGD is a welcome advance in the field of in vitro fertilization. PCR provides performance advantages over other amplification techniques, including, for example, reduction in time required for amplification. In addition, while MDA can be accurate for genotyping, it has been determined to be less accurate for copy number analysis—there is more noise and less accuracy in karyotype providing a greater chance of false positives and negatives (Treff et al. Fertility and Sterility, 88; S231 (2007).

Standard techniques for DNA extraction, isolation, amplification and purification, for enzymatic reactions involving DNA ligase, DNA polymerase, restriction endonucleases and the like, and various separation techniques are those known and commonly employed by those skilled in the art, and may be used as appropriate. A number of standard techniques are described in Miller (ed.) 1972 EXPERIMENTS IN MOLECULAR GENETICS, Cold Spring Harbor Laboratory, Cold Spring Harbor, N.Y.; Old and Primrose, 1994 PRINCIPLES OF GENE MANIPULATION, 5th ed., University of California Press, Berkeley; Schleif and Wensink, 1982 Practical Methods in Molecular Biology; Glover (Ed.) 1985 DNA CLONING: VOLS. I AND 11, IRL Press, Oxford, UK; Harnes and Higgins (Eds.) 1985 NUCLEIC ACID HYBRIDIZATION, IRL Press, Oxford, UK; and Setlow and Hollaender 1979 GENETIC ENGINEERING: PRINCIPLES AND METHODS, Vols. 1-4, Plenum Press, New York City, the entire contents of which are incorporated by reference herein.

Human genome information is available from many public genomic databases familiar to one of skill in the art, such as GenBank from the National Center of Biological Institute (NCBI, and may be accessed as necessary to perform the methods of the present invention.

WGA and Microarray PGD of IVF Embryos

Euploid cells have 22 pairs of autosomes and 1 pair of sex chromosomes. Each pair of chromosomes contains one member from the father and one member from the mother. During early cell division, however, while complete pairs of chromosomes should replicate and migrate from parent to daughter cells, sometimes only one chromosome (monosomy) or an extra chromosome (trisomy) will result. This error is then repeated as the cells continue to grow and divide.

Most of these genetic errors result in embryos which are not viable. They will not implant and generally will not be carried to term. Interestingly, it is only the least severe of these genetic copying errors which can result in a live birth. And yet, such live births can have heartbreaking, long-term consequences for the child suffering from, for example, Down's Syndrome which is a defect because of monosomy or trisomy at chromosome 21, Klinefelter's syndrome (XXY), or Turner's syndrome (XO).

Thus, an important aspect of the present invention is a method of in vitro fertilization that allows an appropriate professional to accurately predict the genetic normalcy, or karyotype, of an IVF embryo (and thus rank or grade these IVF embryos for possible transfer) by using limited quantities of embryonic material. It is contemplated herein that accurate analysis of IVF embryos for possible transfer, including, but not limited to, identifying those embryos which look the most likely to implant and result in a normal child, and those that will most likely not implant, can maximize the chance of a normal pregnancy and reduce the number of embryos that must be transferred for possible implantation and thus reduce the number of unwanted multiple births.

According to the methods of the present invention, a method of in vitro fertilization which includes PGD and selection of IVF embryos for possible transfer may be accomplished from a single cell biopsy using whole genome amplification (WGA) and microarray analysis. While WGA has been attempted before to amplify limited quantities of nucleic acid, the possibility of the introduction of errors associated with this technique has been of concern. As reported herein, the reliability of WGA is ensured in the present invention by evaluating the concurrence of SNP information across all 24 chromosomes (22 pairs of autosomes and 2 sex chromosomes). Specifically, it has been determined that where there is a concurrence of 90% or greater, in another embodiment, 92% or greater, and in still another embodiment, 94% or greater, of about all 250,000 or more SNPs across the entire 24 chromosome genome, the copy number data can be considered robust and reliable for use in PGD. A high degree of concordance across the entire genome assures that the amplification fidelity is good, i.e., that there was no bias and that the amplified data is a true representation of the original unamplified genome. If concurrence/concordance is significantly below these levels, however, there is an unacceptable level of uncertainty in the correctness of the amplification data and thus these data should not be used for PGD. See Example 1.

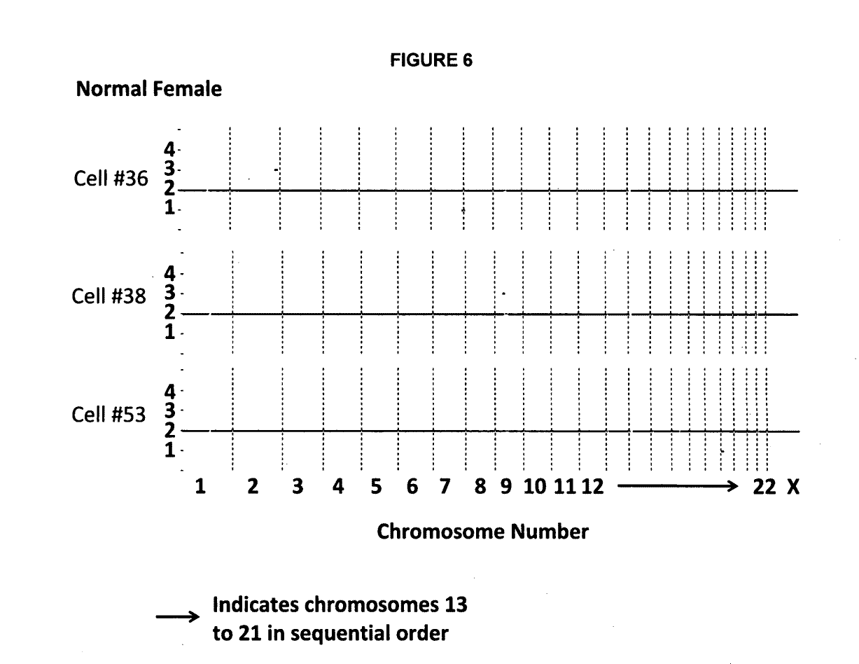

While the degree of copy number concurrence across the entire genome alone may be indicative of the reliability of the data for use in PGD, in some cases, a genome-wide SNP analysis may not provide a sufficient basis to decide whether or not to transfer a particular fertilized embryo. For example, as shown in FIG. 6, a cell (cell number 36) was tested for a normal female. The SNP copy number analysis of the entire genome is represented by the horizontal line at number 2 along the y-axis, indicating that there are two chromosomes, one each from the father and mother for each of the chromosomes. The numbers along the x-axis indicate the chromosome numbers.

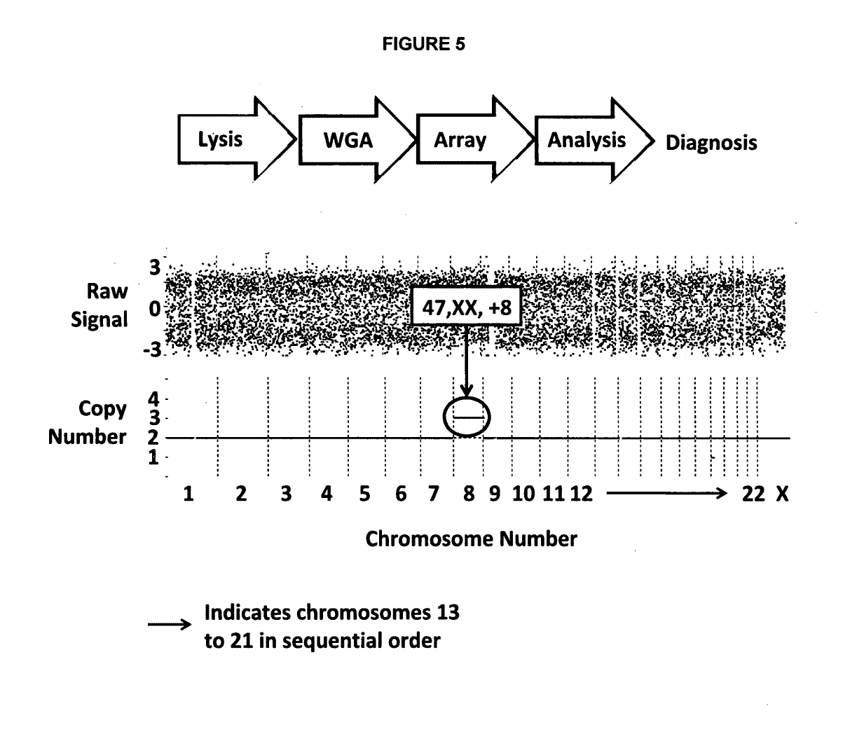

This line is actually made up of over 262,000 dots, each one representing an individual SNP from the microarray. This is better illustrated by the “Raw Signal” information found in, for example, FIG. 5 for a different patient. FIGS. 5 and 6 can be computer generated using standard software such as Copy Number Analysis Tool (CNAT) 4.0.1, available from Affymetrix and are familiar to one of skill in the art. WGA copy number analysis for cell 36 shows better than a 94% concurrence. Indeed, copy number concurrence probably approaches 99% or more.

However, in the second chromosome and in the seventh chromosome, a small group of data shows at 3 and at respectively (along the y-axis). It is unclear whether this small data set at chromosomes 2 and 7 indicate the presence of an extra chromosome or a missing chromosome. Thus, the question basically becomes one of whether or not, despite the overall high concurrence of the copy number analysis across the entire genome, the data is reliable and predictive for the embryo. Such data could be outlying data of no importance or it could indicate that the amplification and/or hybridization process was insufficiently robust. The latter is less likely because of the very high concordance of the WGA analysis across all 24 chromosomes. Unfortunately, however, this data could also mean that a particular embryo is unlikely to be viable.

Thus, as contemplated herein, in order to resolve any doubt regarding the predicted health and possible viability of the embryo, the characterization of ploidy status of individual chromosomes may be typically performed. In this example, individual information for those particular chromosomes (2 and 7) is considered to determine whether or not the cell is aneuploid, e.g., whether it has one copy (monosomy) or three copies (trisomy) of those chromosomes. Thus, with regard to the copy number concordance data for a particular chromosome, according to the methods of the present invention, it is contemplated herein that a concordance of better than about 50% of the SNPs indicates that the data may be used in PGD to predict that an IVF embryo chromosome is normal (euploid) or abnormal (monosomy or trisomy), as the case may be. Thus, if DNA isolated from an IVF embryo is analyzed and a chromosome displays a concordance of 50% or better, for example, and that data for that chromosome indicates trisomy, than that determination would be considered accurate and useful for PGD of this embryo. Otherwise, the data could be indeterminate. In another embodiment, a concordance of 70% or better is deemed reliable for PGD as part of a method of in vitro fertilization. This type of determination can be done on any particular chromosome for PGD, but typically is performed for each chromosome.

It is contemplated herein that the methods of the invention may be used to detect whole chromosome aneuploidy. It may also be used to diagnose embryos from parents who are known carriers of balanced translocations. In the case of balanced translocations, a concordance in a particular region of a chromosome of about 50% or more, and in another embodiment, about 70% or more, indicates that the data accurately indicates a translocation or not, as appropriate.

In addition, since the process described herein involves DNA amplification and because nearly all single gene disorders are diagnosed through a method involving DNA amplification, it is also possible to incorporate single gene disorder testing into the protocol described herein. This can be done for any single gene disorder using methods of DNA analysis that are familiar to one of skill in the art. It is contemplated herein that the methods of the present invention allow for the first time a method of in vitro fertilization comprising PGD with the ability to perform single gene disorder screening in parallel with aneuploidy and translocation screening since previous methods of aneuploidy and translocation screening involve methodologies, other than DNA amplification, e.g., FISH. An example of how a single gene disorder can be evaluated in parallel with aneuploidy and translocation screening is shown in Example 6 below.

In addition to performing whole genome copy number analysis and characterizing the ploidy status of individual chromosomes as discussed above, it is further contemplated herein that the in vitro fertilization methods of the present invention may include a qualitative assessment of chromosomal heterozygosity. Specifically, this comprises identifying either a possible loss or gain in chromosomal heterozygosity as explained in detail below.

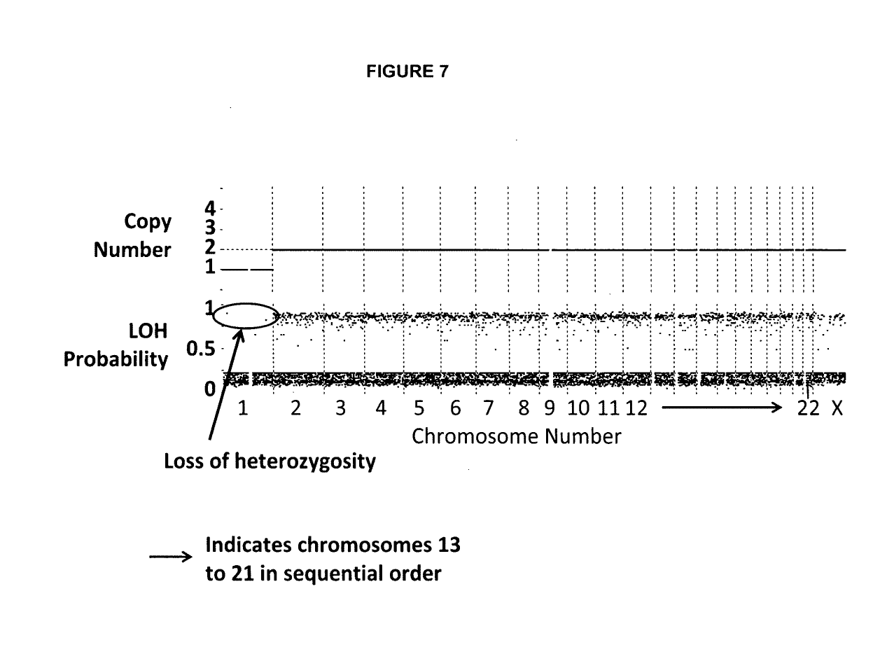

As understood by one of skill in the art, assuming that a chromosome is obtained from both mother and father, most of the genetic information on a particular chromosome should follow Mendelian inheritance rules. However, there are times when particular sequences of DNA, and in particular, selected base pairs, are different. Where the genetic information is matching, it is said to be homogenous or homozygous. For example, as shown in FIG. 7, as indicated by the denser line at the bottom of the lower representation (between about 0.00 and 0.25 along the y-axis), most of the individual points representing SNPs fall in the homozygous category. In contrast, the top, more scattered and diffuse line between about 0.75 and about 1.00 along the y-axis represents SNPs which correspond to heterozygous base pairs. As would be expected, in most instances, there is a fair amount of both, but most SNPs are homozygous.

For example, a pair of chromosomes on which all the SNPs match can be represented by AA, one A from each chromosome, or BB, one B from each parent's chromosome. Where these two match, they are homozygous and, as most of the base pairs and complementary DNA from the father and mother will match, there are far more homozygous data points than heterozygous data points. Where the two are different, one should expect to see a data point for A from one chromosome and B from the other. This is heterozygosity.

However, in an embryo that has only one copy of a chromosome (i.e., chromosomal monosomy) instead of the normal complement (i.e. disomy), all of the SNPs associated with that chromosome will be A or B. They could never be A and B. Thus, there should be a near complete loss of heterozygosity with regard to this chromosome. This is illustrated by the almost complete lack of data in the heterozygous line for chromosome 1 in FIG. 7.

If an embryo possesses three copies of a chromosome (i.e., chromosomal trisomy) instead of two, the presence of an extra chromosome increases the possibility of there being a combination of A and B, and the degree of heterozygosity seen in the embryo would be expected to increase.

Thus, a consideration of the degree of heterozygosity can be a useful step in grading and/or ranking an embryo for PGD and possible transfer by confirming a prediction of monosomy or trisomy.

With regard to the methods of the present invention, e.g., analysis of loss or gain in heterozygosity as part of a method of in vitro fertilization which includes PGD, the methods are not necessarily conducted using all possible SNPs in a genome, but use those which are said to be “informative”. “Informative SNPs” in this instance are those which meet specific “call criteria”, specifically, a stringency of 0.33 or less and in some instances 0.01 or less. As understood by one of skill in the art, identification of informative SNPs with regard to each chromosome and assignment of a proper weight is an important step and may be determined according to conventional methods. For example, it is possible, using genetic information publically available from any of the human genome projects, for one of skill in the art to determine the expected rate of difference to be observed for a given SNP. For example, if, in a given population, there is a relatively high degree of difference for a particular SNP, then such a SNP can be highly diagnostic and is weighted more heavily. Where differences are rarely seen in a given population, it is presumed that the difference seen in a given chromosome or copy number analysis is an artifact of the technique and this is given relatively little weight. The weighting is proportional to the degree of variability, with the higher weights being provided for more variable SNPs. Parental DNA can also be used to determine what to expect from the genomic analysis of embryonic DNA using Mendelian inheritance rules.

Thus, as reported herein and described in detail it is contemplated herein that for the first time, a reliable method of in vitro fertilization which includes PGD using nucleic acid isolated from a single embryonic cell and which comprises whole genome amplification of said nucleic acid, is possible. Specifically, this combination of single-cell WGA and genome-wide SNP microarrays may be reliably used for in vitro fertilization methods incorporating PGD of all 24 chromosomes wherein said method comprises the steps of ensuring at least a 90% SNP copy number concurrence, ensuring a 50% or greater concurrence for a each individual chromosome, and also, optionally, performing a properly weighted heterozygosity analysis to confirm predicted monosomies or trisomies.

Genetic Fingerprinting

Advances in assisted reproduction technologies (ART) have produced significant increases in delivery rates over the last 20 years. However, as discussed above, more than one-half of all babies born in the USA after in vitro fertilization (IVF) are from multiple gestations. Development of laboratory techniques which would allow precise assessment of the reproductive potential of individual embryos has therefore become the most important challenge of contemporary human embryology. One of the major methodologic limits to development of diagnostics of reproductive potential has been a need to know with absolute certainty if an embryo with a particular developmental, genomic, proteomic, or metabolomic marker actually implants and develops into a healthy infant. In the case of a multiple embryo transfer followed by the implantation of a single embryo (the most common scenario), it is not possible to know with certainty which embryo implanted. Single embryo transfer may overestimate the predictive value of a marker of interest as a result of the inability to control for numerous and significant variables, such as follicular stimulation, sperm quality, culture media, laboratory conditions, endometrial receptivity, and embryo transfer technique. Thus, the ability to simultaneously transfer multiple embryos in a single transfer and then know with a high degree of certainty which embryos did and did not implant would be an enormously valuable investigative tool.

When multiple embryos are transferred together, they are typically subjected to the same environmental conditions. That is, they come from a single cohort of developing follicles following a single gonadotropin stimulation, have been inseminated from a single prepared semen sample, develop in identical laboratory conditions, and undergo the exact same transfer into the same endometrium. This eliminates many of the nonembryonic variables which might affect outcome and provides the most precise opportunity for evaluating markers of, or interventions to improve, embryonic reproductive competence.

Thus, as further contemplated herein, and as described in detail in Example 7, the methods of the instant invention permit an accurate 24 chromosome genetic normalcy assessment and thus may be used to create a “genetic fingerprint” of an IVF embryo. Data indicate that by creating such fingerprint, these techniques can be used virtually conclusively to uniquely identify from among the group of transferred IVF embryos, the one (or more) that ultimately implants and, ideally, results in the birth of a healthy child. Thus, by testing the genetic information of a child after a healthy live birth, one is able to correlate that child with a particular implanted IVF embryo. Such immediate feedback offers numerous advantages including, for example, confirming that it was a transferred IVF embryo that implanted and was responsible for the live birth. It also permits one of skill in the art to confirm that the assumptions and observations made concerning the predicted health and viability of that embryo which contributed to the selection of the particular embryo for transfer were correct. For example, it may turn out that, based on a particular selection criteria, the embryo considered most likely to implant consistently does not do so. Instead, based on that criteria, it is actually the second or third most highly ranked embryo which is consistently implanting. As contemplated herein, according to the methods of the present invention, one can reevaluate the underlying assumptions used for the selection and transfer criteria and, possibly, modify them accordingly to further increase the robustness of the selection process. Of course, it will be appreciated that many factors can influence the chances for a healthy, live birth which have little or nothing to do with the capabilities of the embryos selected, and such factors are familiar to one of skill in the art and may be taken into consideration in this analysis.

As further contemplated herein, the invention permits aneuploid chromosome specific fingerprinting which can identify the parental origin of embryonic genetic aneuploidy and this information may be of value with regard to future clinical treatment decisions. For example, in the situation where aneuploidy is evident based on copy number (CN) and loss of heterozygosity (LOH) analysis, the origin can be determined by evaluating the embryonic aneuploid chromosome genotypes at positions where the parental genotypes are homozygous for the opposite allele. If, for example, the embryo inherited only one chromosome (monosomy), then the genotypes for these particular SNPS will be most similar to the parent which actually contributed a chromosome, and less similar to the parent which failed to contribute a chromosome. When applied to a trisomy chromosome, the similarity will be higher for the parent who contributed an extra chromosome and lower for the parent who contributed only one chromosome. When applied to an entire cohort of embryos, this information may be useful in determining if there is a significant contribution to aneuploidy from one parent or the other. This might lead the physician to recommend a sperm or oocyte donor. This technique can also be applied to all chromosomes in the embryo independent of whether euploidy is observed by CN and LOH analyses. Chromosomes which display significantly unequal similarity to one parent or another may represent aneuploid chromosomes that were unidentified by CN or LOH analysis. Alternatively, these chromosomes may represent uniparental disomy (UPD), where one parent contributed two chromosomes instead of one. These situations have been documented to occur and can lead to phenotypic abnormalities.

The methods of the present invention can also comprise a further technique used in addition to or instead of the copy number based techniques described above. This technique utilizes copy number variance. In performing copy number variance analysis, instead of looking for SNPs, wherein single nucleotides are different or polymorphic, one looks to those regions of the chromosome where both parent's DNA are the same. Thus, instead of looking for variation between chromosomes, one judges the intensity. As understood by one of skill in the art, although heterozygosity analysis is not performed when detecting copy number variance, the technique looks at far more data points, and can be very accurate.

It is also understood that, as many regions of the human genome are devoid of SNPs, such regions typically have not been evaluated for copy number variance using SNP microarrays. New advances in commercial microarrays, however, have led to the recent development of probes for these regions by supplementing SNP probes with copy number variant probes (e.g., Affymetrix SNP6.0 GeneChip). These commercially available probes are designed to quantify a region of the DNA devoid of a SNP, and thus may be used to detect copy number variance according to the methods of the present invention.

All patent applications, patents, literature and references cited herein are hereby incorporated by reference in their entirety.

In practicing the present invention, many conventional techniques in molecular biology and recombinant DNA are used. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y., Affymetrix Copy Number Analysis Tool (CNAT) 4.0 Workflow Document; and GeneChip® Mapping 500K Assay Manual.

Also incorporated by reference herein are the following:

Treff et al., Accurate 23 Chromosome Aneuploidy Screening in Human Blastomeres Using Single Nucleotide Polymorphism (SNP) Microarrays, Fertility & Sterility volume 88, page S1 (2007);

Treff and Scott, Genome-wide chromosome aneuploidy assessment on single cells using two types of arrays—SNP based arrays are more accurate and less variable than BAC arrays, Fertility and Sterility volume 88, pages S87-S88 (2007);

Treff et al., Single cell whole genome amplification technique significantly impacts the accuracy and precision of microarray based 23 chromosome aneuploidy screening, Fertility and Sterility volume 88, page S231 (2007);

Treff et al., Single-blastomere whole-genome DNA fingerprinting results in unequivocal embryo identification—a powerful new clinical and diagnostic tool, Fertility and sterility volume 88 pages S3-S4 (2007);

Scott et al., Prospective, Randomized, Blinded, and Paired Analysis of 24 Chromosome Microarray PGD (mPGD) VS 9 Chromosome Fish PGD (fPGD) in Dispersed Cleavage Stage Human Embryos: mPGD has Superior Consistency, Fertility and Sterility volume 90, page S306 (2008);

Miller et al., Reanalysis of Day 3 Fish (fPGD) Abnormal Embryos Which Fully Blastulated: 24 Chromosome Microarray PGD (mPGD) Demonstrates a High Rate of Genetic Normality and Low Rate of Mosaicism Fertility and Sterility volume 90, page S48 (2008);

Scott Jr. et al., Microarray Based 24 Chromosome Preimplanation Genetic Diagnosis (mPGD) is Highly Predictive of the Reproductive Potential of Human Embryos: A Prospective Blinded Nonselection Trial; Fertility and Sterility volume 90, pages S22-S23 (2008);

Miller et al., Blastocyst Formation Rates in Chromosomally Normal Versus Abnormal Embryos as Analyzed by 24 Chromosome Microarray-Based Anueploidy Screening (MPGD) Fertility and Sterility volume 90, page 5346 (2008);

Levy, et al., The Accuracy and Consistency of Whole Genome Preimplanation Genetic Diagnosis (PGD): A Comparison of Two Independent Methods—Microarray PGD (mPGD) and Comparative Genomic Hybridization (CGH) Fertility and Sterility volume 90, page S305 (2008);

Tao et al., Fetal DNA Fingerprinting of DNA Isolated From the Peripheral Maternal Circulation at 9 Gestational Weeks Allows Precise Identification of Which Embryos Implanted Following Multiple Embryo Transfer, Fertility and Sterility volume 90, page S81 (2008);

Treff et al., Characterization of the Source of Human Embryonic Aneuploidy Using Microarray-Based 24 Chromosome Preimplanation Genetic Diagnosis (mPGD) and Aneuploid Chromosome Fingerprinting Fertility and Sterility volume 90, page S37 (2008);

Su et al., Robust Embryo Identification Using First Polar Body (1st PB) Single Nucleotide Polymorphism (SNP) Microarray-Based DNA Fingerprinting, Fertility and Sterility volume 90 pages S82-S83 (2008);

Chung et al., Reprogramming of Human Somatic Cells Using Human and Animal Oocytes, Cloning and Stem Cells volume 11, number 2, pages 213-223 (2009) and supplementary Tables 1-3;

Treff et al., A Novel Single-Cell DNA Fingerprinting Method Successfully Distinguishes Sibling Human Embryos, Fertility and Sterility 2009 (in press).

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

### EXAMPLES

### Example 1

### SNP Based Microarray Analysis Allows Accurate Karyotyping of Single Cells

In view of the limitations of fluorescent in situ hybridization (FISH), including the inability to study all chromosomes, and the technically challenging and tedious procedures associated with high resolution comparative genomic hybridization (CGH), we set out to develop and validate a comprehensive single cell 24 chromosome aneuploidy screening method using SNP microarrays for preimplantation genetic diagnosis (PGD).

An in vitro fertilization method which incorporates a whole genome amplification (WGA) and SNP-based microarray paradigm that may be used to provide accurate single cell 24 chromosome aneuploidy screening for PGD is described below:

Evaluation of Single Cells with Known Karyotypes:

First, embryonic cells were lysed after biopsy in a PCR tube. Single cells were loaded into individual tubes and were randomized and blinded. The single cell genomic DNA was then amplified over 1 million fold using WGA performed using a modification of the GenomePlex system (Sigma-Aldrich) as described with the exception that the DNA is treated with a different lysis buffer prior to amplification (See, Example 2, below; and also Treff et al. Fertility and Sterility, 88; S231 (2007)). The amplified DNA was subjected to reamplification after fragmentation and was labeled so that the intensity of 250,000 positions in the genome could be characterized. Microarray analysis was performed on a genome-wide 250K SNP genotyping microarray (Affymetrix). The probes for the microarrays used in this example were designed based on publically available information on the human genome, e.g., from GenBank. The raw signal intensity for each probe was then analyzed using a copy number analysis tool that resulted in a refined dataset (CNAT 4.0.1; Affymetrix) and using a Gaussian smoothing distance of 5 MB to evaluate karyotype. Data for over 250,000 discrete copy number assignments was plotted in linear fashion.

It was to be expected that the data would indicate that most of the SNPS were at copy number state 2, however, where the data points indicated the probes on a chromosome were at copy number 3, for example, it would indicate that the cell had a trisomy karyotype at that chromosome. With regard to the copy number concordance data for a particular chromosome, if a chromosome displays a concordance of 50% or better for copy number 3, than that chromosome would be considered a trisomy, 50% or more of copy number 2 would be a disomy, and 50% or more of copy number 1 would be a monosomy.

In all, we evaluated 72 single cells in the first phase by studying accuracy at three levels. The first of these was the accuracy of assigning copy number state for each of the over 19 million SNPs (approximately 1656 per chromosome). The SNPs were then assembled according to which chromosome they were from in order to obtain a copy number assignment for each chromosome. The chromosomes were then assembled according to the cell they were from in order to determine the full karyotype of the single cell.

Data indicated that 99.2% of the 19 million SNPs were diagnosed correctly. When these were assembled according to which of the 1656 chromosomes they came from, correct assignment in all but 4 chromosomes was observed, giving a 99.8% accuracy for chromosome assessment. The 4 incorrect diagnoses occurred on different chromosomes indicating equivalent diagnostic accuracy for each of the 24 chromosomes. Finally these 4 inaccurate chromosomes were found in three different cells giving an overall accuracy of single cell karyotyping of 95.8%. It should be noted that one of the interesting features of this type of data is that it provides an internal control for amplification fidelity without knowing the true state of the cell being analyzed.

Since the copy number assignment of SNPs should be identical to other SNPs on the same chromosome, the overall concurrence of SNPs can therefore indicate the adequacy of the overall processing of each cell. Data for the overall SNP concurrence for each of 72 cells analyzed indicated that all the cells were above 94% concurrence with the exception of one cell. The distribution of % SNP concurrence clearly showed this sample to be an outlier and it was demonstrated that samples with greater than 94% concurrence could be considered technically adequate. This outlier analysis provided an internal control for determining whether or not the cell was amplified successfully.

In summary, it was concluded that the method worked with greater than 99% accuracy for evaluating single cell SNP and chromosome copy number, that all chromosomes performed with equivalent diagnostic accuracy, and that single cell karyotypes could be determined with 95.8% accuracy.

In addition, it was demonstrated that if a single cell concurrence of greater than 94% was met, the sample could be considered technically adequate. But if the concurrence was below that threshold, the sample could be considered of questionable fidelity.

Validation of the Technique in a Prospective Randomized and Blinded Study of Cells with Known Karyotypes:

The method was evaluated in a blinded study of single cells from cell lines with well characterized karyotypes. (Corriel Cell Lines: GM00321, GM09286, GM02948, GM01359, GM04610, GM03184, GM04435, GM04626, and GM01201). Twenty seven single cells were isolated, randomized and processed using the method previously discussed. Twenty five of the 27 cells demonstrated greater than 94% concurrence and were therefore considered adequate for evaluation.

Results indicated that the method accurately predicted the ploidy status of the cells. For example, data for three single cells which were independently diagnosed as having a copy number state of 2 for each chromosome did indeed originate from a normal female cell line. Additional data which indicated that three single cells had trisomy 18 and monosomy X were confirmed after unblinding to be from the trisomy 18 male cell line. Other results indicated that a single cell which was given a diagnosis of monosomy 21 was in fact isolated from a monosomy 21 female cell line (data not shown).

Thus, in summary, using this method, a 98.7% accuracy of assigning the correct copy number to 7 million SNPs was obtained, 99.4% for assigning over 600 chromosome copy numbers, and when all cells were evaluated a 92.6% accuracy was obtained. In addition, when samples that didn't meet the standard for greater than 94% concurrence were excluded, a 100% diagnostic accuracy for 25 cells and 575 chromosomes was observed.

Evaluation of the consistency of analysis of blastomeres from research embryos: A prospective, randomized, blinded study to investigate the consistency of evaluating 164 blastomeres from 32 embryos was performed. Since the true nature of the embryo karyotype is unknown, accuracy cannot be directly characterized. Instead, the SNP concurrence with other SNPs on the same chromosome and the concurrence of blastomeres from the same embryo can be evaluated. For example, data from 4 cells from one embryo correctly diagnosed the embryo as having monosomy 19, and trisomy 22. In addition, in another example, each of 4 cells from an embryo consistently correctly indicated monosomy 17 in this embryo. In one of the more interesting and encouraging result obtained in this study, data indicated that an embryo had mosaicism, and each abnormality observed was supported by consistent observations in more than one cell. For example, consistent monosomy of chromosome 22 in all four cells was observed, possibly representing a meiotic error. However, a reciprocal abnormality of chromosome 1 was also observed, where 2 cells acquired a loss of chromosome 1, while the 2 other cells acquired a gain of chromosome 1, possibly representing mitotic error that occurred in an early cell division of the embryo. Finally, a consistent loss of chromosome 4 and 14 was observed in 2 cells, and although all of the cells from this embryo were not analyzed, it is possible that some of the unanalyzed cells would have had gains of these chromosomes.

Finally, it is believed that SNP data collected from an embryo may be used to validate a previous diagnosis of monosomy using the rate of heterozygosity. For example, if there is only one copy of a chromosome, there should not be heterozygosity. Indeed, such data is depicted in FIG. 7 which illustrates a complete loss on chromosome 1 but retention of heterozygosity on the remaining chromosomes. Similarly, it is expected that a diagnosis of trisomy may be confirmed in this way. Overall, the experimental data indicated a high rate of concurrence of SNPs with other SNPs on the same chromosome, i.e., 97.6% for the 43 million SNPs evaluated in 164 single blastomeres compared to 99% for the 19 million studied in 72 single cells from cell lines. Thus, according to our methods, research grade blastomeres could be evaluated 94% of the time. An observed 98% rate of concurrence suggested similar accuracy as with cells obtained from known cell lines and a substantial number of consistent changes in blastomeres from the same embryo was found.

The method disclosed hereinabove permits for the first time a comprehensive and accurate whole chromosome aneuploidy screening for all chromosomes in an IVF embryo (i.e., 22 pairs of autosomes in addition to X and Y chromosomes). It may also be used to detect chromosomal abnormalities in embryos from parents known to be carriers of balanced translocations. Representative examples of these particular uses are provided below.

### Example 2

### Experimental Outline for Preimplantation Genetic Diagnosis (PGD) Using Whole Genome Amplification (WGA) and Microarray Technologies

PGD comprising a combination of whole-genome amplification (WGA) and a SNP-based microarray paradigm as described in Example 1 is also described in general below, with regard to a hypothetical set of 10 IVF embryos:

Ten IVF embryos, created and cultured in vitro according to conventional methods, are microscopically confirmed to be undergoing normal development, based on predefined morphological characteristics. On day 3 post-fertilization in the IVF cycle, these 10 embryos would undergo an optimized single blastomere biopsy. That is, each blastomere would be washed in a hypotonic nuclease- and nucleic acid-free solution, placed into a nuclease- and nucleic acid-free 0.2 ml PCR tube in a 2 microliter (ul) volume and delivered to the molecular biology laboratory. Six μl of water and 1 μl of alkaline lysis buffer (200 millimolar (mM) Potassium Hydroxide/50 mM Dithiothreitol) would be added, followed by incubation at 65° Celsius for 10 minutes, then addition of 1 μl of neutralization buffer (300 mM Potassium Chloride/900 mM Tris-hydrochloride/200 mM Hydrochloride pH 8.3/200 Hydrochloride).

Whole genome amplification would then be performed on the lysates, e.g., as recommended by the supplier of WGA4 GenomePlex Single Cell Whole Genome Amplification Kits (Sigma Aldrich), but modified to begin with the “Library Preparation” step as indicated in the manufacturer's instructions. WGA DNA would then be purified, e.g., using GenElute PCR purification columns (Sigma Aldrich), quantified using a spectrophotometer, and normalized to 50 nanograms per μl in a 5 μl volume. The 5 μl of WGA DNA would then be subjected to reamplification and microarray analysis, e.g., following the recommended protocol for NspI GeneChip sample preparation (Affymetrix Inc., “5 day protocol”); or following the recommended protocol for WGA DNA reamplification (WGA3, Sigma Aldrich), automated purification using a liquid handling robot such as the EpMotion 5075VAC (Eppendorf Inc.), followed by resumption of the recommended protocol for NspI sample preparation beginning with the DNA fragmentation step (Affymetrix Inc., “2 day protocol”). The “2 day protocol” would allow performance of PGD using WGA and microarray analysis as contemplated herein to be completed in time to permit embryo selection on day 5 of embryo development and fresh embryo transfer (i.e., transfer of the embryo without first subjecting the embryo to cryopreservation). This procedure could also be performed on polar bodies (1^{st} and/or 2^{nd}) biopsy tissue. The “5 day protocol” would be sufficient for embryos undergoing cryopreservation for a subsequent frozen embryo transfer cycle and could also be performed on polar body (1^{st} and/or 2^{nd}), or trophectoderm biopsy tissue.

### Example 3

### Microarray Based 24 Chromosome Preimplantation Genetic Diagnosis (mPGD) is Highly Predictive of the Reproductive Potential of Human Embryos: a Prospective Blinded Non-Selection Trial

FISH based PGD has not produced the clinical benefit expected. One problem is the lack of data on the reliability of an abnormal result. Even randomized trials are unhelpful as “abnormal” embryos are not transferred. As new technologies are used to study aneuploidy in human embryos, it is critical to determine both negative and positive predictive values (−PV and +PV). This study assesses the −PV and +PV of mPGD results for clinical outcome. Patients were aged 21-40 and underwent IVF per routine. Embryos were cultured and selected for transfer per routine. Each embryo was biopsied immediately prior to transfer; 1 cell on day 3 or trophectoderm on day 5. WGA and SNP-based microarray PGD screen for aneuploidy and DNA fingerprinting was performed.

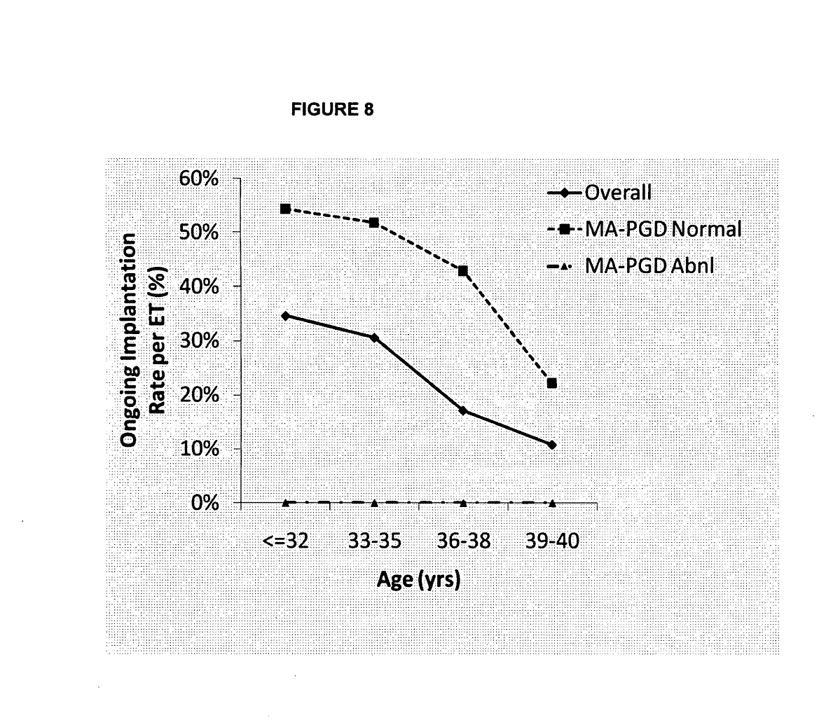

Pregnant patients had blood collected at 9 weeks gestation for fetal DNA isolation and fingerprinting. (Fetal DNA may also being attained after delivery or clinical loss). The specific reproductive potential of each embryo was determined. No implantation equates to no reproductive potential. 100% implantation proves each embryo possessed true reproductive potential. In those transfers wherein a portion of embryos sustained implantation, fingerprinting was done to determine which had potential. Implantation rates for normal and abnormal embryos were compared via contingency tables. Of 100 patients participating in the study, evaluable results were available on 51 at time of assay. These patients had 128 embryos transferred with 18 pregnancies and 36 gestational sacs. Microarray PGD (mPGD) results were highly predictive of ongoing implantation (normal vs abnormal; 42.9% vs 0%; P<0.001). All mPGD abnormal embryos either failed to implant (26; 84%) or implanted and miscarried (5; 16%). Thus the −PV was 100% in all age groups. The +PV was excellent. Implantation rates were highly correlated with % of mPGD normal embryos transferred (P<0.01) (FIG. 8). Other pregnancies were too early for sampling DNA at time of assay. mPGD was highly predictive of embryonic reproductive potential. In the future, mPGD abnormal embryos may safely be discarded as they do not possess significant reproductive potential. That some normal embryos did not implant confirms that factors other than aneuploidy contribute to the inefficiency of human ART.

### Example 4

### Analysis of First IVF Babies Born after Rapid 24 Chromosome Embryo Aneuploidy Screening and Fresh Embryo Transfer

As described hereinabove, microarray based 24-chromosome aneuploidy screening can be successfully applied to single blastomeres in time for fresh blastocyst transfer and result in the delivery of healthy babies. Rigorous technical and clinical validation of a method for comprehensive 24 chromosome aneuploidy screening as described hereinabove was previously performed, including analysis of more than 4,000 oocyte and embryo biopsies and delivery of 51 healthy babies. This protocol was developed to allow for more rapid analysis and availability of data from blastomere biopsy in time for fresh embryo transfer on day 5. This was done by making two modifications to the previously described protocol. First, after WGA of the blastomere, the WGA DNA was processed without purification and by re-amplification with the WGA3 kit (GenomePlex, Sigma Aldrich) rather than through the restriction digest protocol from Affymetrix. This was done in 3 parallel reactions of 150 ul each in order to obtain approximately 90 micrograms of WGA DNA. Purification was then done overnight using the EpMotion 5075 automated liquid handling instrument rather than by manual processing with the conventional Affymetrix protocol. The EpMotion also automated the incubation with fragmentation and labeling buffers (similar to the manual method described in the Affymetrix protocol) so that the next morning the sample would be ready for loading onto the microarray for overnight hybridization. The hybridized chip was then washed, stained, and scanned for analysis and results on day 5 of embryo development and in time for fresh embryo transfer without cryopreservation.

IRB approval and patient consent was obtained in order to proceed with rapid 24 chromosome aneuploidy screening in indicated patients.

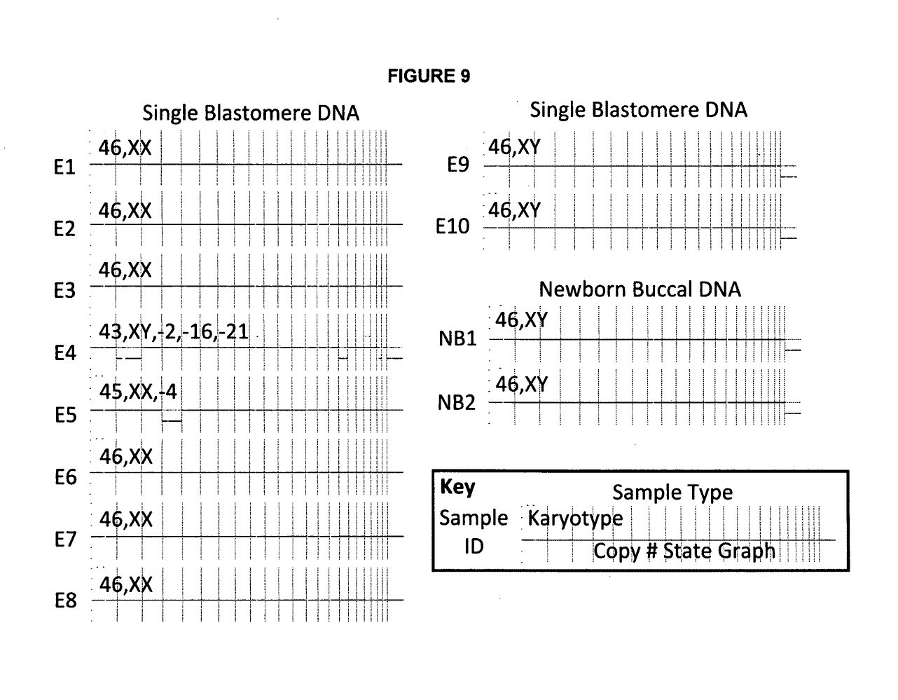

Newborn buccal DNA was collected to confirm embryo identity by microarray DNA fingerprinting as described hereinabove and to generate a molecular karyotype of the newborns. A single blastomere from each of 49 embryos was evaluated from 6 treatment cycles in 5 patients (mean maternal age 43.2 and a range of 34.7 to 46.8 years). Five of the six cases either had aneuploidy in all available embryos or the euploid embryos arrested in extended culture. Only one case led to an embryo transfer in which 8 of the 10 embryos were euploid for 24 chromosomes (6 female and 2 male) and two were aneuploid (43, XY, −2, −16, −21 and 45, XX, −4) (FIG. 9). Interestingly, one of the abnormal embryos in this case would have been diagnosed as normal by the most comprehensive FISH methods currently available. Of the remaining euploid blastocysts, 2 arrested, 4 were cryopreserved, and 2 were replaced, resulting in delivery of two healthy babies with normal karyotypes. Results indicate that it is possible to evaluate aneuploidy of 24 chromosomes on single blastomeres from human embryos in time for a fresh embryo transfer. This is believed to be the first report of delivery of healthy babies after rapid 24 chromosome embryo aneuploidy screening.

### Example 5

### Development and Validation of Comprehensive Triple Factor PGD: Simultaneous 24 Chromosome Aneuploidy, Translocation Derivative, and Single Gene Disorder Screening from a Single Biopsy

Previous methods of aneuploidy screening in PGD have not allowed for parallel single gene disorder screening. For example, using FISH, the DNA is not amplified and the cell is thus consumed in the method for other purposes. In contrast, this example demonstrates how Applicants' method allows for the first time single gene disorder screening, to be performed in parallel with aneuploidy screening in PGD.

A patient with a balanced translocation and Alagille syndrome 46, XX, t(2:20)(q21;p12.2) presented for PGD. She had a 2.36 MB deletion including the JAG1 locus near the translocation. PGD for this case needs to distinguish normal embryos from those with balanced translocations since the latter would still have Alagille syndrome. The goal of this study was to develop a methodology which allows simultaneous screening for translocation derivatives, the deletion related to the single gene disorder, and 24 chromosome aneuploidy screening (AS).

Microarray-based 24-chromosome aneuploidy screening was done using the validated protocol as described herein above. Using this methodology, 121 discarded embryos from 10 patients with various reciprocal balanced translocations were studied and compared to FISH balanced translocation screening results. The ability to accurately determine single gene disorder inheritance was established by evaluating single cells from cell lines and affected embryos from individuals with single gene disorders. Following validation, triple factor PGD was applied to the patient with Alagille syndrome.

Trophectoderm (TE) biopsies were obtained from 6 blasts. Half of the TE biopsy was evaluated by FISH for the microdeletion, translocations, and aneuploidy of chromosome 18, X, and Y. The remaining TE was evaluated by Q-PCR for the microdeletion, and by microarray based screening for the microdeletion, translocations, and 24-chromosome aneuploidy. 2 of the blastocysts were diagnosed as carriers of the maternal balanced translocation and affected by Alagille syndrome, and 4 were unbalanced or had aneuploidy for one or more chromosomes. Q-PCR, MA, and FISH based microdeletion, translocation, and aneuploidy analyses were 100% consistent.

Despite a lack of unaffected euploid embryos available for transfer, the patient has initiated another treatment cycle. This technology represents the only validated method available for comprehensive triple factor PGD.

### Example 6

### Single-Cell DNA Fingerprinting Method to Distinguish Sibling Human Embryos

We have applied DNA fingerprinting techniques to discriminate unrelated individuals from a single somatic cell nuclear transfer-derived blastomere (Chung et al., Cloning Stem Cells. Published online Feb. 2, 2009 [Epub ahead of print]), but application of DNA fingerprinting in the IVF setting will require the ability to discriminate siblings from a single cell. The purpose of the present study was to develop and validate a highly reliable methodology for sibling embryo DNA fingerprinting which investigators could use in future studies evaluating markers or treatments which may affect reproductive competence.

Materials and Methods

Cell Lines

Human B lymphocytes were obtained from 18 individuals (parents and four offspring from each of three families) through the Coriell Cell Repository (CCR, Camden, N.J.) (repository nos. GM13113, GM13114, GM13118, GM13119, GM13120, GM10858, GM10859, GM11870, GM11871, GM11872, GM11875, GM07554, GM07554, GM07440, GM07441, GM07555, and GM07556). Cell lines were cultured as recommended by the supplier (CCR). Single cells were removed using a 100 mm stripper tip and pipette (Midatlantic Diagnostics, Mount Laurel, N.J.) under a dissecting microscope and placed in a nuclease-free 0.2 mL polymerase chain reaction (PCR) tube (Ambion, Austin, Tex.) in a volume of 1 mL medium for subsequent whole genome amplification (WGA). Genomic DNA (gDNA) was also obtained from each cell line as described below.

Embryos

Two sets of embryonic cells were obtained for this study. The first set comprised 72 blastomeres which were derived from 12 abnormally developing day 3 embryos (not suitable for transfer) donated by three IVF patients. Forty-seven of these blastomeres were evaluated by microarray analysis. The second set of cells was obtained from 13 embryos from three IVF patients as part of an ongoing clinical trial to determine the predictive value of single-nucleotide polymorphism (SNP) microarray-based 24-chromosome aneuploidy screening. Individual embryos were placed into calcium/magnesium-free human tubal fluid-Hepes media (Sage In vitro Fertilization, Trumbull, Conn.) for embryo biopsy. Embryos were positioned so that a nucleated cell was adjacent to the anticipated biopsy site. A 25-30 mm hole was opened in the zona pellucida with a series of 3-5 single pulses from an infrared 1.48 mm diode laser using a 1 ms pulse duration at 100% power (Hamilton-Thorne Research, Beverly, Mass.). Nucleated blastomeres were removed through the opening by applying gentle pressure with a blastomere biopsy pipette (Humagen, Charlottesville, Va.) to the adjacent intact zona pellucida to eject individual blastomeres. Before processing, the nuclear status of isolated blastomeres from each embryo was verified by light microscopy using Hoffman optics (Nikon). Blastomeres were placed into PCR tubes as described above for lymphocytes. Discarded follicular fluid and sperm or peripheral whole blood were also obtained to develop parental genotypes after isolation of gDNA as described below. All material was collected with patient consent and under Institutional Review Board (IRB) approval.

Chorionic Villi

Dilatation and curettage procedures were performed as a result of clinical pregnancy loss. Chorionic villi were dissected from products of conception (POC) and used to isolate gDNA as described below. A conventional G-banding karyotype report from a commercial laboratory or a comparative genomic hybridization (CGH) report (see method below) was also obtained. All material was collected with patient consent and under IRB approval.

Genomic and WGA DNA

Five to ten milliliters of follicular fluid with resuspended cellular debris, between 3 and 5 million excess sperm, 10 mL peripheral whole blood, approximately 5×10⁶ cells from cell lines, or dissected villi from POC were used to isolate gDNA using the QIAamp DNeasy Tissue kit as recommended for cell cultures (Qiagen Inc., Valencia, Calif.). Lysis was conducted with the addition of 2-mercaptoethanol (Fisher Scientific, Pittsburgh, Pa.) to the lysis buffer for sperm. Single-cell WGA was performed using a Sigma WGA4 GenomePlex Whole Genome Amplification Kit (Sigma Aldrich, St. Louis, Mo.). The WGA DNA was purified using GenElute PCR purification columns as recommended by the supplier (Sigma Aldrich). Genomic DNA isolation and WGA yields were calculated from the concentration as determined using a Nanodrop spectrophotometer (Nanodrop Inc., Wilmington, Del.).

Microarrays

NspI or StyI GeneChip Mapping 500K microarrays were used as recommended by the supplier (Affymetrix Inc., Santa Clara, Calif.). The dynamic model mapping algorithm (Di et al., Bioinformatics 2005; 21:1958-63) was used to assign a confidence score measuring the reliability of each genotype call made (Gtype version 4.1; Affymetrix). The SNP calls were exported as a text file and imported into Excel (Microsoft, Redmond, Wash.) to determine the overall concordance between samples with and without the use of informative SNPs (potential for sibling discrimination) based on parental gDNA genotypes and Mendelian inheritance rules. Molecular karyotyping was performed by copy number analysis of the microarray data using the Copy Number Analysis Tool (CNAT) version 4.0.1 (Affymetrix).

Comparative Genomic Hybridization

Genomic DNA was extracted from chorionic villi and fluorescently labeled using nick-translation. The CGH probes were prepared and washed as previously described (Levy et al. Genet Med 1998; 1:4-12). The fluorescence ratios (green/red) for at least ten of each autosome and seven of each sex chromosome were obtained per slide. The CGH profiles were compared with a dynamic standard reference interval based on an average of normal cases (Kirchhoff et al. Cytometry 1998; 31:163-73). The standard reference interval was scaled automatically to fit the individual test case. The mean ratio profile of each case with 99.9% confidence was compared with the average ratio profile of the normal cases with similar confidence intervals. The genetic diagnosis was assigned using results obtained at the 99.9% confidence interval. A positive finding was considered to be when the confidence intervals of the patient profile and normal averaged profile did not overlap. Digital image analysis was performed using a Cytovision Probesystem and high-resolution CGH software (Applied Imaging Corp., Santa Clara, Calif.).

Statistical Analyses

Receiver operating characteristic (ROC) curves, sensitivity and specificity, box-and-whisker plots, and standard deviations were calculated using Analyse-It (Analyse-It, Leeds, UK) for Microsoft Excel. Student t test was also used for significance analyses in microarray PCR yield, and a chisquares test was used for total percentage heterozygous AB, homozygous AA, and homozygous BB SNP calls.

Ethics

All research material was obtained with patient consent and under IRB approval.

Results

Validation: DNA Discrimination of Sibling Single Cells.

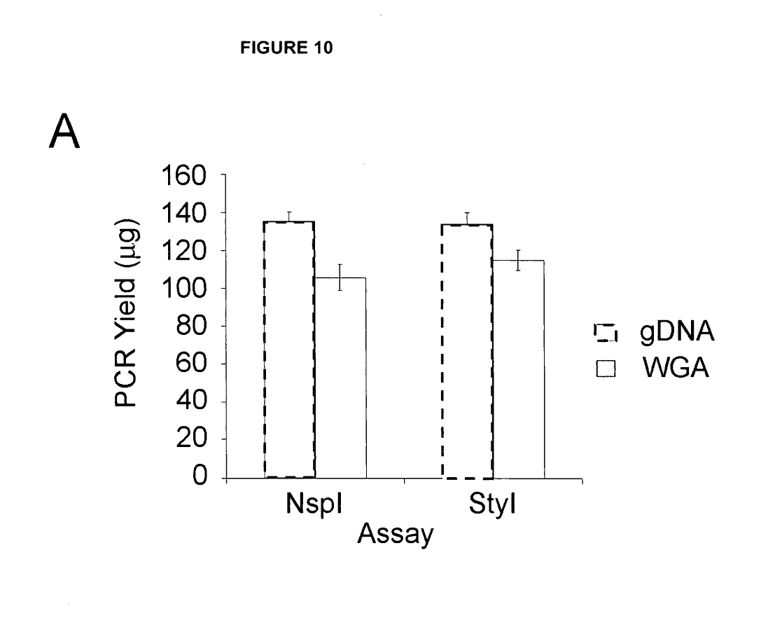

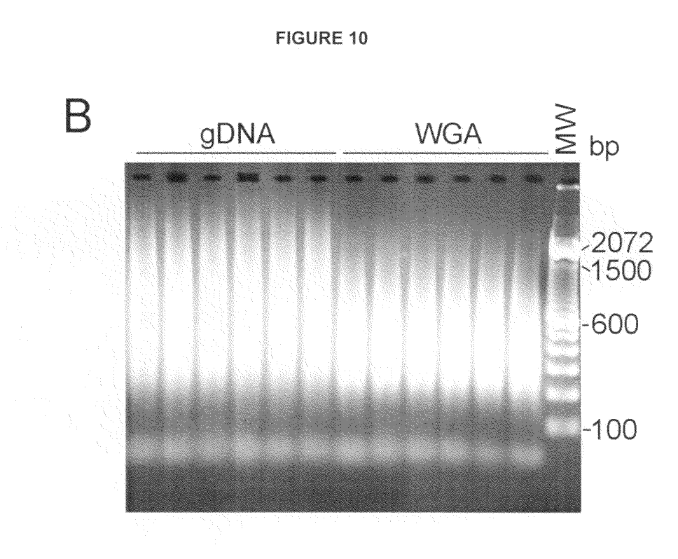

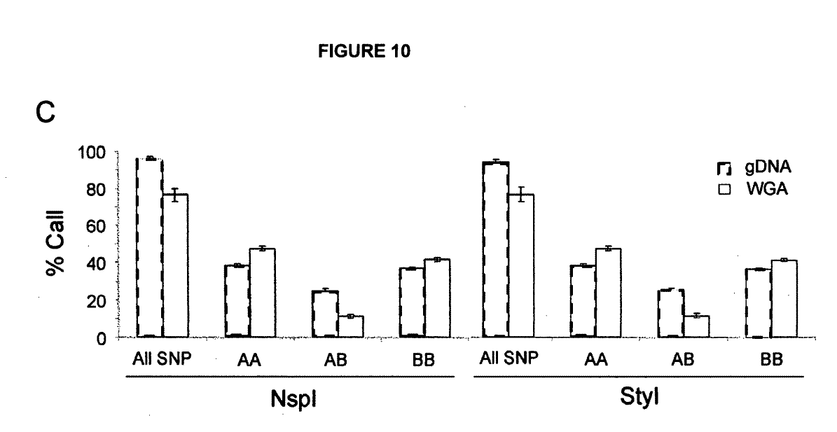

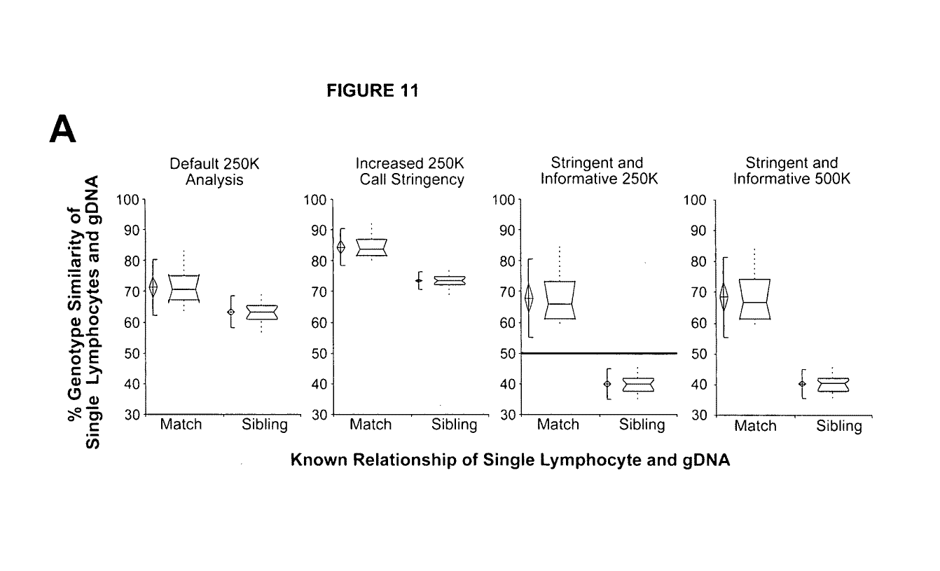

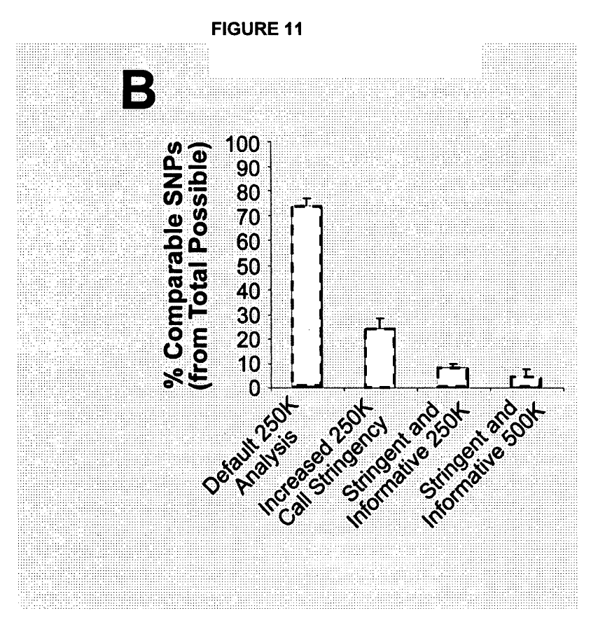

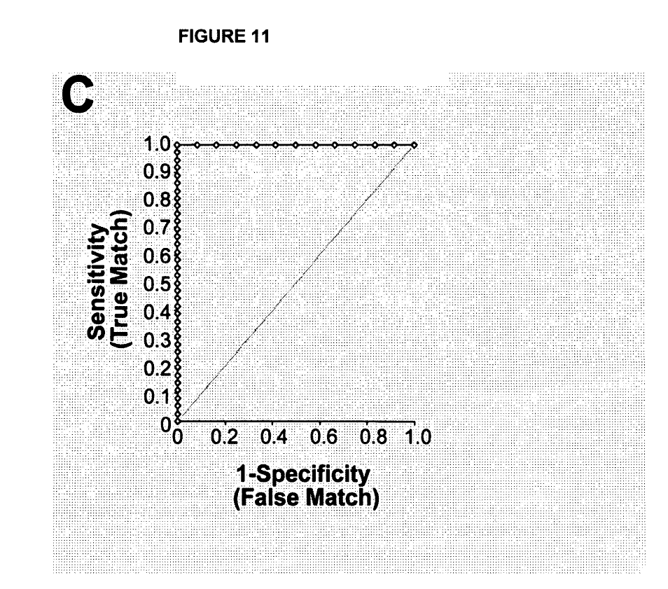

The successful development of an embryo DNA fingerprinting methodology requires that an embryo can reliably be distinguished from its siblings at the single-cell level. We obtained human B-lymphocyte cell lines from 18 individuals, from three sets of unrelated families, each family consisting of two parents and four children. Whole genome amplification was performed on single cells from each child's cell line and yielded an average of 8.3±1.4 mg DNA. A high density oligonucleotide microarray platform was then used to evaluate 500K SNP genotypes in both single cells and corresponding unamplified gDNA samples from each of the three families. The PCR reactions from WGA DNA template yielded less and smaller-sized product than from unamplified gDNA template (FIGS. 10A and 10B). This may be due to the fragmentation of WGA template which doesn't occur during gDNA isolation. In addition, the overall percentage of SNP genotype calls and the number of heterozygous-assigned SNPs were lower from WGA DNA compared with unamplified gDNA (FIG. 10C). This may be due to preferential amplification of one allele or allelic dropout, which has been previously described in other single-cell WGA applications. All possible comparisons of single lymphocytes with either the same individual's gDNA sample or a sibling's gDNA sample indicated that the technique was successful at discriminating between self (matched) and sibling relationships (FIG. 11A, default 250K analysis). To increase the separation of matched and sibling similarities, we increased the confidence level of SNP calls from single cells by adjusting the dynamic model mapping algorithm (Di et al., Bioinformatics 2005; 21:1958-63) from the default analysis setting of 0.33 to a more stringent threshold of 0.01. We found that the increase in stringency improved the level of separation between matched and sibling similarities (FIG. 11A, increased 250K call stringency). To further improve the level of separation between matched and sibling similarities, we evaluated parental gDNA genotypes to identify noninformative SNPs. Noninformative SNPs were defined as those in which both parents were homozygous in any combination (i.e., AA and BB, BB and AA, AA and AA, or BB and BB) and, consequently, SNPs for which all offspring have no opportunity to possess unique genotypes. We found that 36.5±1.5% of the evaluable parental SNPs were noninformative, and that their removal dramatically improved the level of separation between matched and sibling similarities (FIG. 11A, stringent and informative 250K). Results from the second 250K GeneChip (StyI) were similar to the NspI 250K results. However, the combined 500K comparisons didn't improve separation (FIG. 11A, stringent and informative 500K). Overall, an average of 73±4% of the total SNPs were similar between single cells and gDNA samples using the default analysis settings, 24±4% were similar after using higher call stringency for single cells, 8±2% were similar after using higher stringency and only informative SNPs, and 4±3% were similar when high stringency and informative SNPs were evaluated from a total 500K possible SNPs (FIG. 11B). The ROC analysis illustrates a remarkable 100% fingerprinting diagnostic sensitivity and specificity for assigning self and sibling relationships using thresholds between 45.7% and 55.6% (FIG. 11C). Therefore, an average value of 50% was defined as the threshold for assigning self and sibling relationships from a single cell (FIG. 11A, stringent and informative 250K).

Discrimination of Sibling Human Embryo Single Cells

A second set of experiments was designed to demonstrate that this single-cell fingerprinting technology is not only capable of accurate and reliable fingerprinting in lymphocytes, but also in blastomeres, which are the applicable cell type for clinical use. Because unamplified gDNA is not available from the same individuals whence research blastomeres are derived, these experiments involved evaluating the similarity of single blastomeres compared with other single blastomeres from either the same or sibling embryo(s). As a result, this set of experiments used a distinct threshold that is applicable to evaluating single cells relative to other single cells rather than isolated gDNA. To define a threshold comparing a single cell with another single cell for evaluating blastomeres, single lymphocytes were compared with other single lymphocytes either from the same individual (modeling single blastomeres from the same embryo) or siblings (modeling blastomeres from sibling embryos). The ROC curve analysis of this assay demonstrated 100% sensitivity and specificity for thresholds between 66.6% and 78.3% (data not shown). Eighteen discarded embryos (not suitable for transfer) obtained from three sets of patients were dissociated into single cells. Each blastomere was coded so that its identity could be traced back to its originating embryo. Whole genome amplification was performed on each blastomere, and 68 of the 72 blastomeres generated sufficient amounts of DNA for further

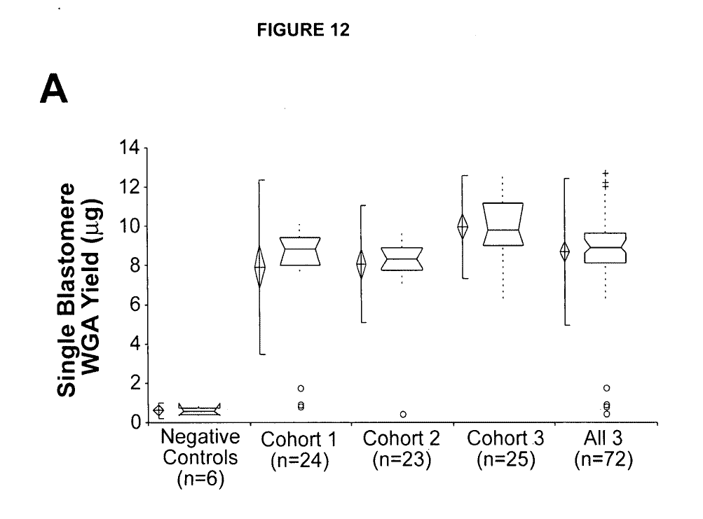

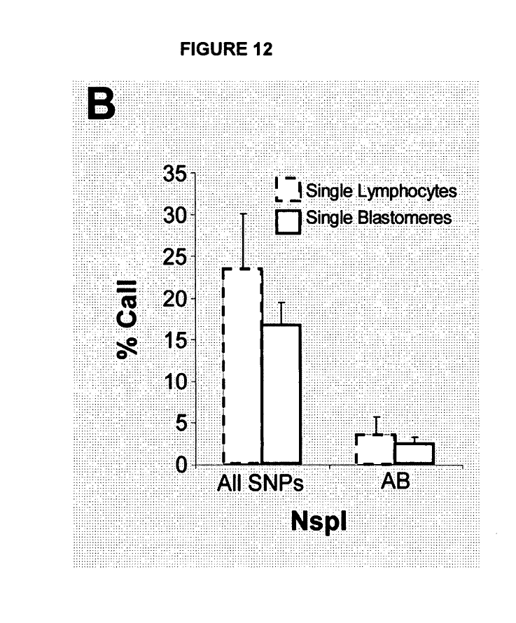

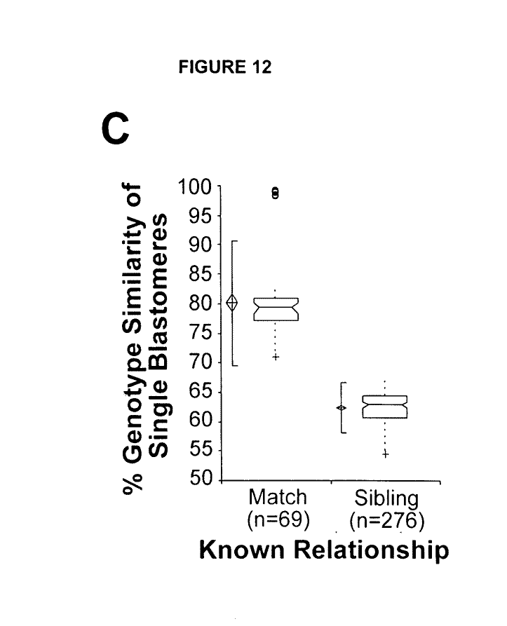

molecular analysis (FIG. 12A). To analyze a similar number of blastomeres and embryos in each of the three patient cohorts, a subset of 47 of these blastomeres were further processed in a blinded fashion by microarray analysis. The overall percentage of SNPs called with high confidence, and the number of SNPs called heterozygous were significantly decreased (P<0.05) in single blastomeres compared with single lymphocytes (FIG. 12B). Although the average number of similar SNPs among single blastomeres was significantly less than the number in single lymphocytes (1.6±0.3% and 2.5±1.0%, respectively; P<0.05), all 47 single blastomere relationships could be predicted with 100% sensitivity and specificity for thresholds, between 67.1% and 71.0% (FIG. 12C). Thus, for each cohort, every blastomere was correctly assigned by DNA fingerprinting back to its originating embryo upon decoding.

Discrimination after Multiple Embryo Transfer

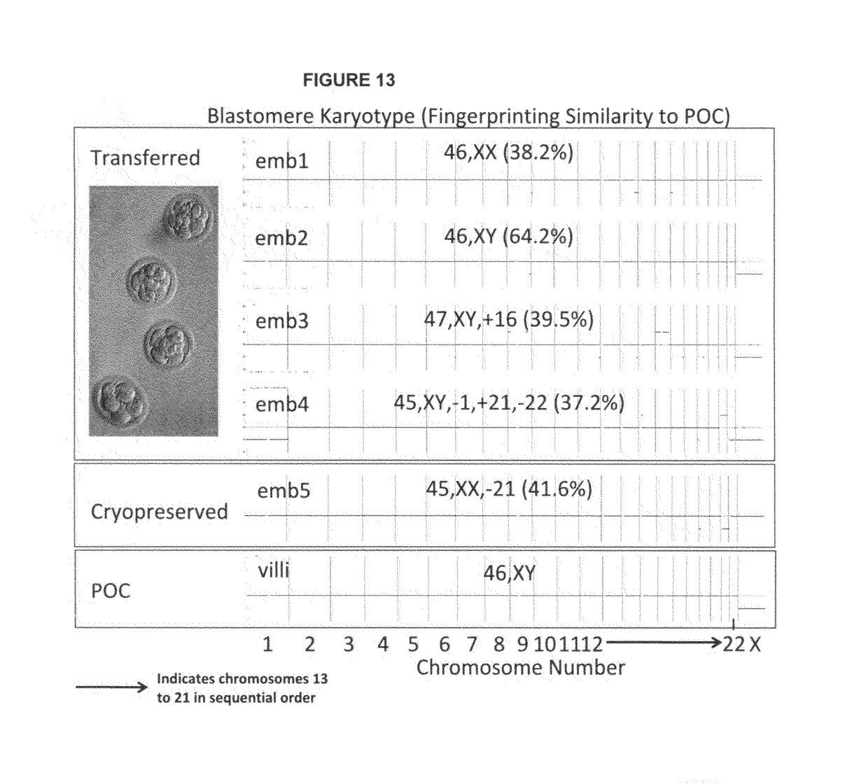

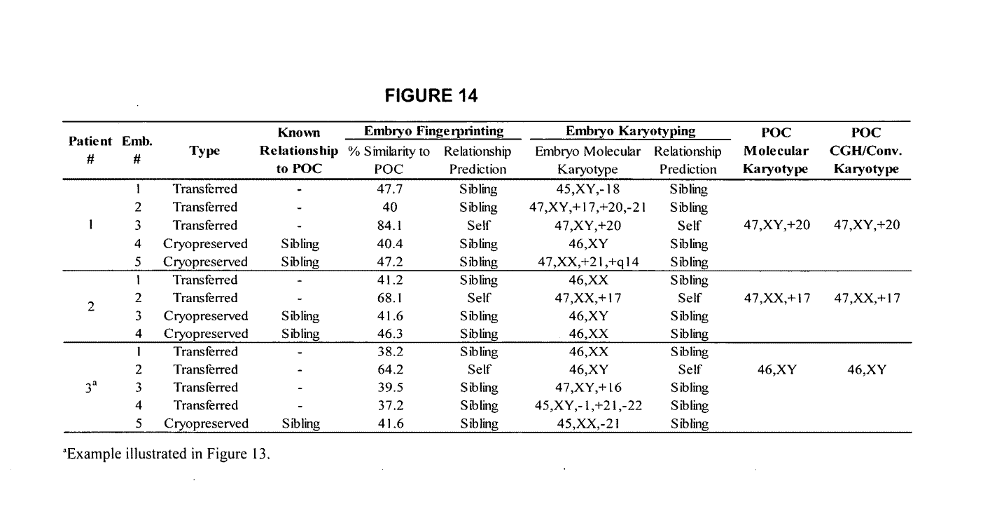

The third set of experiments involved DNA fingerprinting and molecular karyotyping (DNA copy number analysis) of embryos transferred and cryopreserved for three clinical IVF cases where multiple embryo transfer resulted in implantation of a single embryo with subsequent clinical pregnancy loss. The DNA fingerprinting of the POC was performed by microarray SNP analysis, and the fetal karyotype was established by conventional karyotyping and/or molecular karyotyping/high-resolution CGH when conventional karyotyping was not possible. These results were compared with the original single-cell microarray-based fingerprint and molecular karyotype for each embryo transferred or cryopreserved. In each case, the molecular karyotype, conventional karyotype, and CGH results of the POC matched only one of the molecular karyotypes of a transferred embryo (example shown in FIG. 13). In each case, using a 50% threshold of similarity as previously defined in the present study, fingerprinting comparison of the POC with all of the corresponding transferred embryos identified only one match (self), and the molecular karyotype of the matched embryo concurred with the karyotype determined in the POC (FIG. 14).

Percentage similarities of transferred embryos assigned as siblings by fingerprinting were similar to percentages obtained for cryopreserved embryos (known siblings). In addition, percentage similarities were consistent with values obtained in the lymphocyte model system described above (FIG. 11A). These results further validate the clinical applicability of SNP microarray-based embryo fingerprinting.

Discussion

These data demonstrate that it is possible to reliably use DNA fingerprinting to distinguish the embryonic origin of single cells, and that the specificity is sufficient to distinguish single cells from singling and sibling embryos. This is an important new tool which will empower scientists and clinicians to consistently identify which embryo among those which were transferred actually implanted and proceeded through to clinical development. The screening strategy successfully distinguished single cells from a sibling when compared in a blinded fashion with gDNA. This is the paradigm which will be used clinically. The genetic fingerprint from a single cell biopsied from each transferred embryo can be compared with genomic DNA obtained at the time of antenatal screening, from cord blood at the time of delivery, or from buccal swabs of the newborn infant. The accuracy of this technique was 100% for sibling/self discrimination.

Although the focus of earlier fingerprinting studies was not necessarily accuracy of distinguishing siblings, it is possible that the high level of accuracy in the present study stems from significantly increasing (by several orders of magnitude) the number of genetic loci evaluated in parallel, thereby overcoming the limitations associated with allelic dropout or preferential amplification. For example, previous studies used eight short tandem repeats (STRs) to distinguish siblings. The assignment of a match between an embryo and newborn was never based on more than one locus, nor was the accuracy of the technique established when applied to single cells from sibling or self cell lines. (Jones et al. Hum Reprod 2008; 23:1748-59). In contrast, we evaluated over 262,000 SNPs, where the assignment of a match was based on no less than 2,000 informative SNPs. We also established a 100% accuracy level on single cells and blastomeres from sibling or self cell lines and embryos, respectively. This accuracy was accomplished by combining single-cell WGA, which has recently seen considerable advances in preimplantation genetic diagnosis applications (see, e.g., Coskun and Alsmadi Prenat Diagn 2007; 27:297-302) with genome-wide SNP microarrays for the first time.

SNP-based fingerprinting has also been used in the forensic analysis of highly degraded material because of improved applicability to small DNA fragments compared with STRs, which target larger-molecular-weight DNA. This may be beneficial in the analysis of single blastomeres, which may have a high degree of DNA fragmentation. We also found that use of parental DNA SNP profiles, to identify informative loci for distinguishing offspring, dramatically improved the discriminatory power of the technique. Although no technique should ever be considered to be error free, the precision of these results is high. In fact, this diagnostic tool is powerful enough to allow comparison of a single cell from one embryo to other single cells obtained from either the same embryo (a match) or from sibling embryos (a nonmatch). This represents a significantly more stringent standard, because the error induced by the imprecision of WGA occurs before both microarray analyses, functionally doubling the potential for error.

This technique may not, in and of itself, change the way ART is practiced. Instead, it will empower clinicians and scientists to evaluate questions with better experimental designs. One simple example is optimization of studies evaluating different culture conditions. Earlier studies have evaluated the influence of different culture conditions by comparing two groups of patients with different embryo culture conditions. With DNA fingerprinting, comparisons of different media, different CO₂ concentrations, or different O₂ concentrations can now be evaluated with every patient serving as her own control. The two-pronuclear embryos from an individual patient would be randomly assigned to two groups. One of the groups might be cultured in one medium preparation and another in a second type of medium. The single best embryo from each group would be selected. These two embryos (one from each group) would then be biopsied and transferred. If the patient gets pregnant with twins, the groups had equivalent outcomes. Similarly, if she does not become pregnant then the two embryos had the same outcome. However, in those cases where one embryo implanted, it is possible to use fingerprinting on DNA obtained from the conceptus (or from the baby if done after delivery) and determine which embryo implanted.

By applying this paradigm to a series of patients, it will be possible to evaluate the resulting data with a McNemar chi square and determine if one medium is outperforming the other. Because each patient serves as her own control, smaller sample sizes are typically needed. At the risk of stating the obvious, the quality of the data would also be superior. Because each patient's data are compared only with herself, there is no potential for stimulation dynamics, other laboratory conditions, endometrial receptivity, or transfer quality to affect results. In addition to aiding in this type of intervention study, marker development, such as genomic, transcriptomic, proteomic, and metabolomic approaches to embryo assessment, could be enhanced through DNA fingerprinting-controlled experimental designs.

In summary, the availability of reliable DNA fingerprinting from single cells is likely to have a significant effect on research in reproductive medicine. Expected improvements in experimental design, reduction in the number of subjects that are required to address important embryology and laboratory issues, and greater precision in distinguishing outcomes should empower investigators to complete more relevant and statistically powerful studies in shorter periods of time.

1. A method of in vitro fertilization wherein said method includes preimplantation genetic diagnosis of an IVF embryo comprising the steps of:
a) biopsying said IVF embryo to remove one or more cells from said embryo;
b) extracting nucleic acid from said one or more cells;
c) performing whole-genome amplification of said nucleic acid;
d) gathering genetic information for said IVF embryo comprising performing the following substeps (i) and (ii):
i) determining the copy number of one or more SNPs for all 24 chromosomes in said IVF embryo; and
ii) determining the copy number of all 24 chromosomes in said IVF embryo; and optionally
iii) determining the presence of a loss or gain in chromosomal heterozygosity in said IVF embryo;
e) predicting the genetic normalcy of the IVF embryo based on the genetic information obtained in (d) if the genetic information collected in (d)(i) indicates at least 90% SNP copy number concurrence, and the genetic information collected in (d)(ii) indicates at least a 50% chromosomal copy number concordance and where applicable, the genetic information collected in (d)(iii) indicates neither a gain nor loss of heterozygosity and
(f) selecting one or more IVF embryos for transfer based on the determination made in step (e).

2. The method of claim 1 wherein the biopsy is of a polar body from the embryo.

3. The method of claim 1 wherein the biopsy is of trophectoderm from the embryo.

4. The method of claim 1 wherein the biopsy is of blastomeres from the embryo.

5. The method of claim 1 wherein the biopsy is taken from the embryo at day 0 to day 6 of development.

6. The method of claim 1 further comprising transferring one or more IVF embryos selected according to step (f).

7. The method of claim 6 wherein said one or more IVF embryos are transferred within 48-72 hours of embryo biopsy.

8. The method of claim 6 wherein said one or more IVF embryos are frozen prior to transfer.

9. The method of claim 6 wherein said one or more IVF embryos are not frozen prior to transfer.

10. The method of claim 6 wherein the genetic information determined for each individual IVF embryo is recorded prior to transfer.

11. The method of claim 1 wherein substeps (d)(i), (d)(ii) and (d)(iii) are performed.

12. A method of identifying the genetic characteristics of an IVF embryo likely to result in successful embryo transfer and live birth comprising
(a) gathering genetic information from a child resulting from a successful pregnancy achieved according to the methods of claim 10; and
(b) comparing said genetic information to the recorded data for each IVF embryo transferred, in order to identify the IVF embryo that implanted and resulted in the live birth; and
(c) determining whether assumptions and observations made which lead to the selection of said embryo for transfer were correct.

13. The method of claim 10 further comprising identifying the specific IVF embryo which implanted and resulted in a successful pregnancy and live birth of a child comprising
(a) gathering genetic information from the child;
(b) comparing said genetic information to the genetic information recorded for each IVF embryo transferred, and
(c) using the recorded genetic information for each transferred embryo as a genetic fingerprint by which the child may be linked to the correct transferred embryo.

14. A method of in vitro fertilization which permits the identification of transferred and implanted IVF embryo(s) comprising performing in vitro fertilization according to the method of claim 1 and further comprising gathering genetic information from a resulting child or children and comparing with genetic information previously recorded for each transferred embryo.
